(19) United States
(12) Patent Application Publication
(10) Pub. No.: US 2023/0137510 A1
(43) Pub. Date: May 4, 2023

(54) BREATHING ASSISTANCE APPARATUSES AND/OR COMPONENTS THEREOF AND/OR USES THEREOF

(71) Applicant: Fisher & Paykel Healthcare Limited, Auckland (NZ)

(72) Inventors: Kevin Peter O'Donnell, Auckland (NZ); Charles Grady Cantrell, Auckland (NZ); Christopher Harding Campbell, Auckland (NZ); Anton Kim Gulley, Auckland (NZ); Robert Stuart Kirton, Auckland (NZ); David Martin Russell, Auckland (NZ); James Alexander Michael Revie, Auckland (NZ); Benjamin Wilson Casse, Auckland (NZ); Hamish Chan, Auckland (NZ)

(21) Appl. No.: 17/755,622

(22) PCT Filed: Nov. 4, 2020
(86) PCT No.: PCT/IB2020/060335

§ 371 Date: May 3, 2022

Related U.S. Application Data
(60) Provisional application No. 62/930,515, filed on Nov. 4, 2019.

(51) Int. Cl. A61M 16/00; A61M 16/08; A61M 16/10

(52) U.S. Cl. CPC A61M 16/026; A61M 16/0875; A61M 16/1005; A61M 2205/505

(57) ABSTRACT

This disclosure enables displaying of an intuitive and engaging enquiry on a touchscreen of a medical device (e.g., breathing apparatus, breathing assistance apparatus) that a patient is already using (e.g., in-home medical device). Since the patient is already accustomed to using the medical device for medical treatment control, the patient, who may be unwell, is more likely to access the enquiry and complete the enquiry. Further, making the enquiry intuitive and engaging encourages the patient to regularly interact with the enquiry, while ensuring that the enquiry is not overly tedious to complete. Responding to the enquiry can be made mandatory by refraining from activating or preventing activation of a component of the apparatus until a predetermined set of responses has been received.

### TECHNICAL FIELD

The present disclosure relates to various breathing assistance apparatuses and/or components thereof and/or uses thereof.

### BACKGROUND ART

Breathing assistance apparatuses are used in various environments, such as hospital, medical facility, residential care, or home environments, to deliver a flow of gas to users or patients. The breathing assistance apparatuses come in various forms, such as a standalone humidifier apparatus, a continuous positive airway pressure (CPAP) apparatus, a ventilator, or a high flow apparatus.

The breathing assistance apparatuses typically have one or more accessories, such as a breathing conduit, and a patient interface, such as a cannula or mask, for delivering gases to a patient. The conduit enables gases to be delivered from the housing of the breathing assistance apparatus to the patient. For example, the apparatus may be placed on a floor or other support surface, and the patient may be in a bed. The breathing assistance apparatus may have a recess for receipt of a humidifier liquid chamber. The liquid chamber will receive liquid from, for example, a flexible liquid bag that delivers liquid to a humidifier liquid chamber via one more tubes. Alternatively, the liquid chamber can be removed and refilled as required. The recess will contain a heater plate to heat the liquid chamber, to humidify gases passing through the liquid chamber. The humidified gases are then delivered to the patient.

In this specification, where reference has been made to external sources of information, including patent specifications and other documents, this is generally for the purpose of providing a context for discussing the features of the present disclosure. Unless stated otherwise, reference to such sources of information is not to be construed, in any jurisdiction, as an admission that such sources of information are prior art or form part of the common general knowledge in the art.

### SUMMARY

In an aspect the disclosure relates to a breathing assistance apparatus, configured to supply breathable gasses to a patient, comprising a user interface that comprises a touchscreen that is configured to present a plurality of user health queries and a plurality of user input elements via which user inputs are received, and

wherein supply of breathable gasses from the breathing assistance apparatus to the patient is conditional on the breathing assistance apparatus receiving a predetermined plurality of user inputs.

In a further aspect the disclosure relates to the delivery of ventilation to a patient through the use of a breathing assistance apparatus, configured to supply breathable gasses to the patient, and

wherein the breathing assistance apparatus comprises a user interface that comprises a touchscreen that is configured to present a plurality of user health queries and a plurality of user input elements via which user inputs are received, and

wherein the breathing assistance apparatus refrains from activating or prevents activation of a component of the breathing assistance apparatus until a predetermined plurality of the user inputs have been received.

In a further aspect the disclosure relates to a breathing assistance apparatus comprising
a source of breathable gas or gasses;
a breathing conduit that conveys the breathable gas to a patient via a patient interface;
a touchscreen located on or in the breathing assistance apparatus;
a controller to control the breathing assistance apparatus operations, including activation and operation of the breathing assistance apparatus,

wherein the controller is configured to:
a) request the touchscreen to display a plurality of user health queries and a plurality of user input elements via which user inputs are received,
b) refrain from activating or prevent activation of a component of the apparatus until a predetermined plurality of the user inputs have been received.

In a further aspect the disclosure relates to the use of a breathing assistance apparatus to provide respiratory support for a patient, comprising:
a) providing a breathing assistance apparatus comprising
   a touchscreen;
   a breathing conduit that conveys breathable gas to a patient via a patient interface;
   a controller to control the breathing assistance apparatus operations, including activation and operation of the breathing assistance apparatus, the controller comprising an electronic processor and a memory in communication with the electronic processor;
b) providing instructions executable by the electronic processor that when executed by the electronic processor cause the controller to
   request the touchscreen to display a plurality of user health queries and a plurality of user input elements via which user inputs are received,
   refrain from activating or prevent activation of a component of the apparatus until a predetermined plurality of the user inputs have been received.

The breathing assistance apparatus may comprise a network interface to send the predetermined plurality of user inputs to a patient and device management platform (e.g. a server) via the network interface.

The breathing assistance apparatus may comprise a housing, a flow generator in the housing for generating a breathable gas, and an outlet in flow communication with the breathing conduit.

In a further aspect the disclosure relates to breathing assistance apparatus that provides a flow of breathable gases to a user, the breathing assistance apparatus comprising

a housing;

a user interface disposed on an upper surface of the housing such that it is visible and accessible, the user interface comprising a colour touch screen and/or one or more buttons;

a source of breathable gas;

a controller adapted to control the delivery of breathable gases to a user, the controller in electronic communication with the user interface to control the user interface to present two or more queries sequentially in response to receiving a start up input at the user interface, the two or more queries presented as a combination of words and/or indicia and/or graphics and/or selectable buttons,

the user interface receiving a response to each query, the response to each query communicated to the controller;

the controller configured to process the response to each query and/or transmit the response to each query to a remote electronic device, and

the user interface configured to present the two or more queries prior to presenting an operative parameter selection screen to the user, wherein the operative parameter selection screen allows the user to select one or more operative parameters.

In a further aspect the disclosure relates to a breathing assistance apparatus that provides a flow of breathable gases to a user, the breathing assistance apparatus comprising

a housing;

a user interface disposed on an upper surface of the housing such that it is visible and accessible, the user interface comprising a colour touch screen and/or one or more buttons;

a source of breathable gas;

a controller adapted to control the delivery of breathable gases to a user, the controller in electronic communication with the user interface to control the user interface to present two or more queries sequentially in response to receiving a start up input at the user interface, the two or more queries presented as a combination of words and/or indicia and/or selectable buttons,

the user interface receiving a response to each query, the response to each query communicated to the controller;

the controller configured to process the response to each query and/or transmit the response to each query to an external or remote service or platform,

the controller configured to
lock access to any other modes or functions until the controller receives a response to each query, or
restrict access to an operative mode until the controller receives response to each query, or
disable operation of the flow generator and humidifier until a response to each query is received; and

wherein the queries presented to the patient include
a first query related to how a user is feeling and presenting a plurality of discrete selectable responses,
a second query related to the condition of a user's throat and the user interface presenting a plurality of selectable indicia, each indicia representing (or corresponding) to a response, and
a third query related to a breathing or breathing rate of a patient.

In one example a response is received by the controller by a user (i.e. a patient) interacting with the touch screen and/or the one or more buttons.

A response may constitute an answer to the query.

A response may be entered by performing a gesture on the touch screen and/or touching or pressing or interacting with specific portions of the touch screen.

A response may be entered by a user (i.e. patient), by pressing or manipulating one or more buttons in a specific combination or specific order.

A response to a query may be entered by a user by interacting with the touch screen (e.g. performing a gesture etc.) and pressing or manipulating the one or more buttons in a specific combination.

The source of breathable gases may be a flow generator that is part of the breathing assistance apparatus.

The flow generator may be controlled by the controller to control the delivery of breathable gases to a user.

The flow generator may be a pump or a blower or compressor or any other suitable unit that is configured to generate a pressurized flow of gases.

The breathing assistance apparatus may comprise a humidifier.

The humidifier may comprise at least a heater plate and a humidification chamber configured to hold a humidification liquid (for example as a humidification liquid chamber) .

The chamber may be configured to be removably positioned on the heater plate such that the heater plate can heat the contents of the chamber.

The controller may be configured to control the heater plate to heat the contents of the chamber.

The humidifier may be located downstream of the source of breathable gases and the flow of breathable gases is delivered to the humidifier for humidification.

In a further aspect the disclosure relates to a breathing assistance apparatus that comprises:

a housing having a fluid inlet and a fluid outlet;

a flow generator located within the housing downstream of and in fluid communication with the fluid inlet;

a humidifier located within the housing downstream of and in fluid communication with the flow generator and upstream of and in fluid communication with the fluid outlet, the humidifier including a heater;

a touchscreen located on the housing;

a network interface located within the housing; and

a controller located within the housing and in electrical communication with the flow generator, the humidifier, and the touchscreen, and the network interface, the controller comprising an electronic processor and a memory in communication with the electronic processor, the controller controlling activation and operation of the flow generator, the humidifier, and the touchscreen,

wherein the memory includes instructions executable by the electronic processor that when executed by the electronic processor cause the controller to
activate the heater upon activation of the breathing assistance apparatus,
request the touchscreen to display a plurality of user health queries and a plurality of user input elements via which user inputs are received,
refrain from activating or prevent activation of a component of the apparatus (e.g., controls the apparatus or the flow generator), until a predetermined plurality of the user inputs have been received, and
send the predetermined plurality of user inputs to a patient and device management platform via the network interface.

In a further aspect the disclosure relates to a breathing assistance apparatus that comprises:

a housing having a fluid inlet and a fluid outlet;

a flow generator located within the housing downstream of and in fluid communication with the fluid inlet;

a humidifier located within the housing downstream of and in fluid communication with the flow generator and upstream of and in fluid communication with the fluid outlet, the humidifier including a heater;

a touchscreen located on the housing; and

a controller located within the housing and in electrical communication with the flow generator, the humidifier, and the touchscreen, the controller comprising an electronic processor and a memory in communication with the electronic processor, the controller controlling activation and operation of the flow generator, the humidifier, and the touchscreen,

wherein the memory includes instructions executable by the electronic processor that when executed by the electronic processor cause the controller to
a) request the touchscreen to display a user interface presenting a plurality of requests for user health information and a plurality of user input elements via which the user information is received by the controller as user inputs, and
b) refrain from activating or prevent activation of a component of the apparatus (e.g.

controls the apparatus, the flow generator, or the humidifier), until a predetermined plurality of the user inputs from the user input elements are received.

In a further aspect the disclosure relates to a breathing assistance apparatus comprises:

a housing having a fluid inlet and a fluid outlet;

a flow generator located within the housing downstream of and in fluid communication with the fluid inlet;

a humidifier located within the housing downstream of and in fluid communication with the flow generator and upstream of and in fluid communication with the fluid outlet, the humidifier including a heater;

a touchscreen located on the housing; and

a controller located within the housing and in electrical communication with the flow generator, the humidifier, and the touchscreen, the controller comprising an electronic processor and a memory in communication with the electronic processor, the controller controlling activation and operation of the flow generator, the humidifier, and the touchscreen,

wherein the memory includes instructions executable by the electronic processor that when executed by the electronic processor cause the controller to refrain from activating or prevent activation of a component of the apparatus (e.g. controls of the apparatus, the flow generator, or the humidifier) until a predetermined plurality of user inputs responsive to a plurality of displayed requests for user health information are received via the touchscreen unless the displayed requests for user health information are user bypassed.

Features from one or more embodiments or configurations may be combined with features of one or more other embodiments or configurations. Additionally, more than one embodiment may be used together during a process of respiratory support of a patient.

The queries may be selected from any one or more of a
a) general subjective healthcare query,
b) specific subjective healthcare query,
c) specific objective healthcare query, and
d) any combination of (a) to (c).

The general subjective healthcare query may relate to the patient's general perception of the status of their health.

The specific subjective healthcare query may be a qualitative or quantitative query.

A specific subjective healthcare query may relate to the patient's perception of the status of specific anatomy (e.g. throat) or a specific physiological process (e.g. breathing).

The specific subjective healthcare query may be a qualitative or quantitative query.

A specific qualitative subjective healthcare query may relate to the patient's perception of the status of specific anatomy (e.g. throat) or a specific physiological process (e.g. breathing).

A specific quantitative subjective healthcare query may relate to the patient's perception of the frequency of an action (e.g. coughing).

The specific objective healthcare query may relate to a measurable health parameter (e.g. sputum colour, frequency or amount of medicament use).

One or more queries may be presented as a written query. For example the query may comprise text presented on the user interface.

In one example one or more queries are presented as a combination of a written query and a plurality of selectable answers, wherein the plurality of selectable answers are presented as a geometric shape, picture, icon, indicia, diagram, or writing, or a combination thereof.

In one example the touchscreen may have a resolution of at least 300×150 pixels.

The touchscreen may have a resolution of at least 400×250 pixels.

The touchscreen may have a resolution of at least 480×272 pixels.

The touchscreen may have a resolution of at least 600×400 pixels

The touchscreen may be at least 3.5 inches in diagonal measurement.

The touchscreen may be at least 4 inches in diagonal measurement.

The touchscreen may be 4.2 inches in diagonal measurement.

The touchscreen may be up to 7 inches in diagonal measurement.

The touchscreen may be an OLED or TFT LCD screen.

The touchscreen may comprise a resolution that is high enough to present icons, graphics, colours, indicia, diagrams, text or writing, pictures or images, photographs in visual manner that is easy to see, easy to read and clear. The resolution is high enough to improve legibility of the content provided on touchscreen.

The breathing assistance apparatus may comprise a controller that is configured to determine a change in a physiological condition or a pathology of the patient based on the patient's answers (i.e. responses) to the enquiry.

The patient's data may be delivered to an external or remote service or platform for example, a remote patient and device management platform.

The patient and device management platform may be any one or a combination of a remote device, server, an application, a cloud service or any other suitable hardware and software platform

The external or remote service or remote platform may be a healthcare provider computing system (e.g. a physician server), or an equipment provider system (e.g. a durable medical equipment provider server) or an insurance provider system (e.g. an insurance provider server).

The external or remote service or platform may be configured to determine a change in a physiological condition or a pathology of the patient based on the patient's answers to the enquiry.

The patient and device management platform may be configured to receive responses to the queries and determine a change in the patient's physiological condition or pathology based on the responses.

The patient and device management platform may compare a received response with previous stored responses and determine a change in the patient's physiological condition or a pathology.

The patient and device management platform may be configured to determine a change in the physiological condition or a pathology based on the comparison of a patient response to a patient baseline score (e.g. a baseline score associated with the query).

A determination in the change in the physiological condition or pathology of a patient, may be presented to the patient, a healthcare provider, or both the patient and a healthcare provider.

The controller may be configured to receive a start up input/request via a user interface of the touch screen.

The controller may be configured to communicate two or more queries sequentially to the touchscreen.

The controller may be configured to
lock access to any other modes or functions until the controller receives a response to each presented query, or
restrict access to an operative mode until the controller receives response to each query, or
disable operation of the flow generator and/or humidifier until a response to each query is received.

In a further aspect the disclosure relates to a breathing assistance apparatus comprising

a housing;

a source of breathable gas or gasses;

a breathing conduit that conveys the breathable gas to a patient via a patient interface;

a touchscreen located on the housing;

a controller located within the housing to control the breathing assistance apparatus operations, including activation and operation of the breathing assistance apparatus,

wherein the controller is configured to:
request the touchscreen to display a plurality of user queries and a plurality of user input elements via which user inputs are received,
refrain from activating or prevent activation of a component of the apparatus until a predetermined plurality of the user inputs have been received.

The controller may be in electrical communication with the touchscreen and comprises an electronic processor and a memory in communication with the electronic processor, the controller controlling activation and operation of the apparatus.

The memory may include instructions executable by the electronic processor that when executed by the electronic processor cause the controller to
a) request the touchscreen to display a plurality of user queries and a plurality of user input elements via which user inputs are received,
b) refrain from activating or prevent activation of a component of the apparatus until a predetermined plurality of the user inputs have been received.

The apparatus may further comprise a flow generator, a humidifier located within the housing downstream of and in fluid communication with a flow generator and upstream of and in fluid communication with a fluid outlet, the humidifier including a heater plate, and wherein the controller is in electrical communication with, to control the activation and operation of, the flow generator and the heater plate.

The apparatus may further comprise a network interface, located in the housing and in electrical communication with the controller, to send the predetermined plurality of user inputs to an external or remote service or platform.

The apparatus may further comprise:

a power switch coupled to the controller and that switches between an on-mode and an off-mode, the breathing assistance apparatus is activated based on the power switch switching from the off-mode to the on-mode.

The power switch may be a button, a knob, a dial, a rocker, a toggle, or a lever.

The instructions may cause the controller to request the network interface to send the predetermined plurality of user inputs to server patient and device management platform one-by-one after each of the user inputs is received via the controller.

The instructions may cause the controller to request the network interface to send the predetermined plurality of user inputs to the patient and device management platform after all of the user inputs are received via the controller.

The instructions may cause the controller to request the network interface to send the predetermined plurality of user inputs to the patient and device management platform on a group-basis after a group of the user inputs is received via the controller.

At least one of the user input elements may be included in a group of user input elements corresponding to at least one of the user queries.

The group of user input elements may include at least two user input elements that are visually distinct from each other.

The at least two user input elements may be visually distinct from each other based on color.

The at least two user input elements may be visually distinct from each other based on a shade of color.

The at least two user input elements may be visually distinct from each other based on shape.

The at least two user input elements may be visually distinct from each other based on size.

The group of user input elements may range between 2 and 9.

The group of user input elements may include a group of text strings.

The group of user input elements may include a group of graphics.

At least one member of the group of graphics may have an alphanumeric content extending at least one of therein or thereon.

At least one member of the group of graphics may correspond to an alphanumeric content other than the user health queries, the alphanumeric content is positioned external to the at least one member.

At least one of the user health queries may include a concluding query mark.

The user interface may include a plurality of pages on which the user queries and the user input elements are distributed.

The user interface may include a page presenting the user health queries and the user input elements.

The instructions may cause the controller to repeatedly request the touchscreen to display the user health queries and the user input elements over a period of at least two consecutive days.

The network interface may receive a message from the patient and device management platform, the message is based on the predetermined plurality of user inputs sent to the patient and device management platform via the network interface.

The message may include a video content for output via the touchscreen.

The housing may house a speaker, the message includes an audio content for output via the speaker.

The network interface may be a first network interface, the message is a first message, the housing having a second network interface that sends a second message to a computing device responsive to the first message, the second message is associated with the predetermined plurality of user inputs sent to the patient and device management platform via the first network interface, the second network interface local to the computing device, the computing device is other than the patient and device management platform.

The message may be informative of a user health parameter worsening over a predetermined time period as determined based on at least one of the predetermined plurality of user inputs.

The predetermined time period may be at least two days.

The message may be informative of the user health parameter worsening relative to a baseline.

The message may be informative that two or more health parameters are worsening for at least two days and the patient is therefore deteriorating.

The query may include health queries.

At least one of the user input elements may be included in a group of user input elements corresponding to at least one of the user health queries, wherein the group of user input elements includes an odd number of user input elements, wherein the baseline is associated with a median user input element from the odd number of user input elements.

At least one of the user input elements may be included in a group of user input elements corresponding to at least one of the user health queries, wherein the group of user input elements includes an odd number of user input elements, wherein the baseline is associated with a non-median user input element from the odd number of user input elements.

At least one of the user input elements may be included in a group of user input elements corresponding to at least one of the user health queries, wherein the group of user input elements includes an even number of user input elements, wherein the baseline is associated with a user input element from the even number of user input elements.

At least one of the user input elements may be included in a group of user input elements corresponding to at least one of the user health queries, wherein the group of user input elements includes a first input element that denotes a current position or a non-change state of the health parameter, a second input element that denotes an improvement in the health parameter, and a third user input element that denotes a deterioration of the health parameter.

At least one of the user input elements may be a member of a pair of user input elements corresponding to at least one of the user health queries, wherein the pair of user input elements corresponds to a pair of user inputs that are associated with a pair of data points that are mutually exclusive to each other, wherein the user inputs include at least one member of the pair of user inputs.

The instructions may cause the controller to prevent or preclude skipping the user health queries before activating the flow generator.

The instructions may cause the controller to allow skipping the user health queries before activating the flow generator.

The instructions may cause the controller to request the touchscreen to display a page after the controller receives the user inputs, the page presents a menu to control or activate the flow generator or the humidifier or to input an operational parameter of the flow generator or the humidifier.

The instructions may cause the controller to request the touchscreen to display the user health queries and the user input elements such that at least one of the user health queries or at least one of the user input elements is different between at least two instances of the flow generator being activated over a predetermined time period.

The predetermined period may be at least two days.

The user health queries may include a first query and a second query, wherein the first query precedes the second query, wherein the user inputs include a first user input and a second user input, wherein the first user input precedes the second user input, wherein the first user input corresponds to the first query, wherein the second user input corresponds to the second query, wherein the second query is content-dependent on the first user input.

The first user input and the second user input may be from a same user session.

The first user input may be from a first user session, wherein the second user input is from a second user session, wherein the first user session precedes the second user session.

The controller may receive the predetermined plurality of user inputs before the heater reaches a predetermined temperature.

The user health queries and the user input elements may be displayed upon booting of the controller.

The controller may refrain from activating the flow generator.

The controller may refrain from activating the flow generator upon booting of the controller.

The controller may prevent activation of the flow generator.

The controller may prevent activation of the flow generator upon booting of the controller.

The predetermined plurality of the user inputs may be all of the user inputs.

At least one of the user health queries may relate a user disease progression or a user health condition.

The instructions may cause the controller to allow skipping the user health queries before activating the flow generator such that the user health that are skipped form a plurality of data points that are sent to the patient and device management platform via the network interface.

The controller may be configured request the touchscreen to display a plurality of user queries and a plurality of user input elements via which user inputs are received when the apparatus is in a warm up process, and/or a drying process.

In a further aspect the disclosure relates to a breathing assistance apparatus comprising:
a housing having a fluid inlet and a fluid outlet;
a flow generator located within the housing downstream of and in fluid communication with the fluid inlet;
a humidifier located within the housing downstream of and in fluid communication with the flow generator and upstream of and in fluid communication with the fluid outlet, the humidifier including a heater;
a touchscreen located on the housing; and
a controller located within the housing and in electrical communication with the flow generator, the heater, and the touchscreen, the controller comprising an electronic processor and a memory in communication with the electronic processor, the controller controlling activation and operation of the flow generator, the humidifier, and the touchscreen, wherein the memory includes instructions executable by the electronic processor that when executed by the electronic processor cause the controller to (a) request the touchscreen to display a user interface presenting a plurality of requests for user health information and a plurality of user input elements via which the user health information is received by the controller as user inputs and (b) refrain from activating or prevent activation of a component of the apparatus until a predetermined plurality of the user inputs from the user input elements are received.

The requests for user health information and the user input elements may be displayed upon booting of the controller.

The controller may refrain from activating the flow generator or the heater.

The controller may refrain from activating the flow generator or the heater upon booting of the controller.

The controller may refrain from activating the flow generator.

The controller may refrain from activating the heater.

The controller may prevent activation of the flow generator or the heater.

The controller may prevent activation of the flow generator or the heater upon booting of the controller.

The controller may prevent activation of the flow generator.

The controller may prevent activation of the heater.

The predetermined plurality of the user inputs may be all of the user inputs.

The controller may refrain from activating or prevents activation of the flow generator or the heater until the predetermined plurality of the user inputs from the user input elements are received by the controller unless the requests for user health information and the user input elements are user bypassed.

A breathing assistance apparatus comprising:
a housing having a fluid inlet and a fluid outlet;
a flow generator located within the housing downstream of and in fluid communication with the fluid inlet;
a humidifier located within the housing downstream of and in fluid communication with the flow generator and upstream of and in fluid communication with the fluid outlet, the humidifier including a heater;
a touchscreen located on the housing; and
a controller located within the housing and in electrical communication with the flow generator, the humidifier, and the touchscreen, the controller comprising an electronic processor and a memory in communication with the electronic processor, the controller controlling activation and operation of the flow generator, the humidifier, and the touchscreen, wherein the memory includes instructions executable by the electronic processor that when executed by the electronic processor cause the controller to refrain from activating or prevent activation of a component of the apparatus until a predetermined plurality of user inputs responsive to a plurality of displayed requests for user health information are received via the touchscreen unless the displayed requests for user health information are user bypassed.

The displayed requests for user health information may be displayed upon booting of the controller.

The controller may refrain from activating the flow generator or the heater.

The controller may refrain from activating the flow generator or the heater upon booting of the controller.

The controller may refrain from activating the flow generator.

The controller may refrain from activating the heater.

The controller may prevent activation of the flow generator or the heater.

The controller may prevent activation of at least one of the flow generator or the heater upon booting of the controller.

The controller may prevent activation of the flow generator.

The controller may prevent activation of the heater.

The predetermined plurality of the user inputs may be all of the user inputs.

In a further aspect the disclosure relates to a breathing assistance apparatus comprising:
a flow generator
a humidifier comprising a heater plate configured to heat the contents of the humidification chamber,
a breathing conduit that conveys the breathable gas to a patient via a patient interface;
a user I/O interface;
a controller to control the flow generator and humidifier,
wherein the controller is configured to present a health enquiry comprising one or more queries, when the apparatus is in a warm up process, and/or a drying process.

The health enquiry may be presented by displaying a plurality of user queries and a plurality of user input elements via which user inputs are received.

The apparatus may be configured to activate the warm up process:
a) On startup of the apparatus, or
b) when the user is prompted to enter one or more operative parameters of the breathing assistance apparatus (optionally via a therapy control screen), or
c) when the user begins to enter one or more operative parameters of the breathing assistance apparatus (optionally via a therapy control screen), or
d) once the user has initiated therapy (optionally via a therapy control screen), or
e) when manually activated by a user.

The warm up process may comprises controlling the heater plate to specific temperature,

The specific temperature may be based on one or more temperature set points of the apparatus (optionally the temperature set points are one or more operative parameters of the apparatus).

The specific temperature may be about 35 degrees Celsius.

The apparatus may comprise a heater plate temperature sensor, and the controller controls the temperature of the heater until the heater plate temperature sensor reaches the specific temperature.

The warm up process may comprise controlling the heater plate of the breathing assistance apparatus to control a chamber outlet temperature to a specific temperature.

The chamber outlet may be the outlet of a humidification chamber, and optionally measured in an elbow located after the chamber outlet of the humidification chamber.

Controlling the heater plate may comprise controlling a power provided to the heater plate.

The specific temperature may be based on one or more temperature set points (optionally as one or more operative parameters of the apparatus for therapy) of the apparatus.

The specific temperature may be within about 5 degrees Celsius to about 15 degrees Celsius, or about 10 degrees Celsius, or optionally about 5 degrees Celsius to about 15 degrees Celsius, or about 10 degrees Celsius less than one or more temperature set points.

The one or more temperature set points may be a desired dew point, or a desired patient end temperature.

One or more temperature set points may correspond to a required relative humidity or a required absolute humidity, optionally the relative humidity is about 90% to about 100% or is about 100%.

The warm up process may comprise controlling a heater wire in a conduit attached to the apparatus configured to provide gases to a patient to control a temperature at the end of the conduit to an end of the conduit specific temperature.

The end of the conduit specific temperature may be based on one or more temperature set points (optionally as one or more operative parameters of the apparatus for therapy) of the apparatus.

The end of the conduit specific temperature may be determined by the controller based on the selected one or more temperature set points that represents a desired humidity

The end of the conduit specific temperature may be a predetermined temperature.

The end of the conduit specific temperature may be based on one or more temperature set points, and wherein the one or more temperature set points a desired patient end temperature.

The end of the conduit specific temperature may be within about 2 degrees Celsius to about 5 degrees Celsius, or about 2.5 degrees Celsius of the desired patient end temperature, and optionally, about 2 degrees Celsius to about 5 degrees Celsius, or about 2.5 degrees Celsius less than the desired patient end temperature

One or more temperature set points may comprise a desired chamber outlet temperature, a desired dew point temperature (chamber outlet or at the end of the breathing conduit), or a desired temperature at the end of the breathing conduit.)

The warm up process may comprise the controller deactivating the flow generator.

The warm up process may comprise running the flow generator at a predetermined flow rate or a predetermined flow generator output.

The predetermined flow rate may be lower than a therapeutic flow being provided to the patient.

The predetermined flow generator output may be a motor speed about 1000 RPM to about 3000 RPM or less than about 2000 RPM.

The controller may be configured to activate the drying process:
a) at the end of a therapy session, or
b) when manually activated by a user, or
c) when a standby process has been active for a predetermined amount of time.

A user may indicates via the user I/O interface when the therapy session has ended

The end of therapy may be determined by the detection that a user has taken off the patient interface (and optionally that a predetermined period of time has elapsed.)

The drying process may comprise controlling a heater wire in the breathing conduit while the flow generator provides gases as a set flow rate.

The drying process may comprise controlling a heater plate of the humidifier to a predetermined value, or the heater plate may be deactivated during the drying process.

The heater wire may be controlled by the controller to a predetermined temperature at the end of the patient breathing conduit, or controlled to a predetermined duty cycle or to a predetermined power.

The predetermined duty cycle may be 100%.

The predetermined temperature may be greater than 45 degrees Celsius.

The drying process may be configured to operate for at about 20 minutes to about 40 minutes, or about 15 minutes.

The drying process may comprise controlling the flow generator to provide a predetermined flow generator output, wherein the flow generator output is a motor speed about 1000 RPM to about 3000 RPM or less than about 2000 RPM.

The drying process may comprise controlling the flow generator to provide a predetermined flow rate, wherein the predetermined flow rate is about 10 litres/minute to about 20 litres/minute.

The drying process may be configured to evaporate remaining condensate in the apparatus and/or patient breathing conduit and/or patient interface.

The controller may be configured to refrain from activating or prevent activation of a component of the apparatus until a predetermined plurality of the user inputs have been received.

The controller may refrain from providing therapy until the user inputs have been received and/or the warm-up process is complete.

Based on the answers to the user queries related to one or more health parameters and one or more historic answers to the user queries related to one or more health parameters the controller may be configured to determine a deterioration of patient health, wherein the deterioration of patient health is based on:

a worsening of two or more health parameters for at least two days (optionally from a baseline).

In a further aspect the disclosure relates to a breathing assistance apparatus comprising:

a flow generator

a breathing conduit that conveys the breathable gas to a patient via a patient interface;

a user I/O interface;

a controller to control the flow generator and humidifier,

wherein the controller is configured to display the health enquiry on the user I/O interface, the health enquiry comprising one or more user queries relating to one or more health parameters, wherein each query comprises a plurality of user input elements via which user inputs are received as answers to the user queries, and

wherein based on the answers to the user queries related to one or more health parameters and one or more historic answers to the user queries related to one or more health parameters the controller is configured to determine a deterioration of patient health,

wherein an deterioration of patient health is based on:
a worsening of two or more health parameters for at least two days (optionally from a baseline).

The one or more historic answers to the user queries relating to one or more health parameters are stored locally on the apparatus and/or on a patient and device management platform.

The apparatus and/or a or the patient and device management platform may be configured to notify a user if the deterioration of patient health is determined.

The deterioration of patient health may be additionally based on: a worsening in one or more patient parameter (optionally from a baseline).

The patient parameter may be one or more of: a patient oxygen saturation (optionally measured by at least one patient sensor) and/or a patient respiratory rate (optionally determined from a flow signal from a flow sensor).

The patient parameter may be measured by at least one patient sensor.

The health enquiry may comprise one or more queries related to a COPD condition, and/or one or more queries related to a bronchiectasis condition.

The queries of the health enquiry may be ordered such that queries related to a COPD condition are displayed first, followed by queries related to a bronchiectasis condition.

General health queries may be presented before queries related to a COPD condition and queries related to a bronchiectasis condition.

The apparatus may comprise a humidifier comprising a heater plate configured to heat the contents of the humidification chamber.

The user I/O may be a touchscreen, wherein the touchscreen is at least 3.5 inches in diagonal measurement, or at least 4 inches in diagonal measurement, or 4.2 inches in diagonal measurement.

The health enquiry may be displayed at the beginning of therapy and/or the end of therapy.

The controller may be configured display the health enquiry when the apparatus is in a warm up process, and/or a drying process.

The apparatus may be configured to activate the warm up process:
a) On startup of the apparatus, or
b) when the user is prompted to enter one or more operative parameters of the breathing assistance apparatus (optionally via a therapy control screen), or
c) when the user begins to enter one or more operative parameters of the breathing assistance apparatus (optionally via a therapy control screen), or
d) once the user has initiated therapy (optionally via a therapy control screen), or
e) when manually activated by a user.

The warm up process may comprises controlling the heater plate to specific temperature,

The specific temperature may be based on one or more temperature set points of the apparatus (optionally the temperature set points are one or more operative parameters of the apparatus).

The specific temperature may be about 35 degrees Celsius.

The apparatus may comprise a heater plate temperature sensor, and the controller controls the temperature of the heater until the heater plate temperature sensor reaches the specific temperature.

The warm up process may comprise controlling the heater plate of the breathing assistance apparatus to control a chamber outlet temperature to a specific temperature.

The chamber outlet may be the outlet of a humidification chamber, and optionally measured in an elbow located after the chamber outlet of the humidification chamber.

Controlling the heater plate may comprise controlling a power provided to the heater plate.

The specific temperature may be based on one or more temperature set points (optionally as one or more operative parameters of the apparatus for therapy) of the apparatus.

The specific temperature may be within about 5 degrees Celsius to about 15 degrees Celsius, or about 10 degrees Celsius, or optionally about 5 degrees Celsius to about 15 degrees Celsius, or about 10 degrees Celsius less than one or more temperature set points.

The one or more temperature set points may be a desired dew point, or a desired patient end temperature.

One or more temperature set points may correspond to a required relative humidity or a required absolute humidity, optionally the relative humidity is about 90% to about 100% or is about 100%.

The warm up process may comprise controlling a heater wire in a conduit attached to the apparatus configured to provide gases to a patient to control a temperature at the end of the conduit to an end of the conduit specific temperature.

The end of the conduit specific temperature may be based on one or more temperature set points (optionally as one or more operative parameters of the apparatus for therapy) of the apparatus.

The end of the conduit specific temperature may be determined by the controller based on the selected one or more temperature set points that represents a desired humidity

The end of the conduit specific temperature may be a predetermined temperature.

The end of the conduit specific temperature may be based on one or more temperature set points, and wherein the one or more temperature set points a desired patient end temperature.

The end of the conduit specific temperature may be within about 2 degrees Celsius to about 5 degrees Celsius, or about 2.5 degrees Celsius of the desired patient end temperature, and optionally, about 2 degrees Celsius to about 5 degrees Celsius, or about 2.5 degrees Celsius less than the desired patient end temperature

One or more temperature set points may comprise a desired chamber outlet temperature, a desired dew point temperature (chamber outlet or at the end of the breathing conduit), or a desired temperature at the end of the breathing conduit.)

The warm up process may comprise the controller deactivating the flow generator.

The warm up process may comprise running the flow generator at a predetermined flow rate or a predetermined flow generator output.

The predetermined flow rate may be lower than a therapeutic flow being provided to the patient.

The predetermined flow generator output may be a motor speed about 1000 RPM to about 3000 RPM or less than about 2000 RPM.

The controller may be configured to activate the drying process:
a) at the end of a therapy session, or
b) when manually activated by a user, or
c) when a standby process has been active for a predetermined amount of time.

A user may indicates via the user I/O interface when the therapy session has ended

The end of therapy may be determined by the detection that a user has taken off the patient interface (and optionally that a predetermined period of time has elapsed.)

The drying process may comprise controlling a heater wire in the breathing conduit while the flow generator provides gases as a set flow rate.

The drying process may comprise controlling a heater plate of the humidifier to a predetermined value, or the heater plate may be deactivated during the drying process.

The heater wire may be controlled by the controller to a predetermined temperature at the end of the patient breathing conduit, or controlled to a predetermined duty cycle or to a predetermined power.

The predetermined duty cycle may be 100%.

The predetermined temperature may be greater than 45 degrees Celsius.

The drying process may be configured to operate for at about 20 minutes to about 40 minutes, or about 15 minutes.

The drying process may comprise controlling the flow generator to provide a predetermined flow generator output, wherein the flow generator output is a motor speed about 1000 RPM to about 3000 RPM or less than about 2000 RPM.

The drying process may comprise controlling the flow generator to provide a predetermined flow rate, wherein the predetermined flow rate is about 10 litres/minute to about 20 litres/minute.

The drying process may be configured to evaporate remaining condensate in the apparatus and/or patient breathing conduit and/or patient interface.

The controller may be configured to refrain from activating or prevent activation of a component of the apparatus until a predetermined plurality of the user inputs have been received.

The controller may refrain from providing therapy until the user inputs have been received and/or the warm-up process is complete.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

It should be understood that alternative embodiments or configurations may comprise any or all combinations of two or more of the parts, elements or features illustrated, described or referred to in this specification.

Some embodiments of this disclosure may also be said broadly to consist or comprised in the parts, elements and features referred to or indicated in the specification of the application, individually or collectively, and any or all combinations of any two or more said parts, elements or features, and where specific integers are mentioned herein which have known equivalents in the art to which this disclosure relates, such known equivalents are deemed to be incorporated herein as if individually set forth.

The term “comprising” as used in this specification means ‘including’. When interpreting each statement in this specification that includes the term ‘comprising’, features other than that or those prefaced by the term may also be present. Related terms such as ‘comprise’ and ‘comprises’ are to be interpreted in the same manner.

The term request when used in the context of a controller may refer to the controller sending a signal to a component to instruct the component to perform one or more actions.

As used herein the term ‘(s)’ following a noun means the plural and/or singular form of that noun.

As used herein the term ‘and/or’ means ‘and’ or ‘or’, or where the context allows both.

The disclosure discloses the foregoing and also envisages constructions of which the following gives examples only.

### BRIEF DESCRIPTION OF THE DRAWINGS

Specific embodiments and modifications thereof will become apparent to those skilled in the art from the detailed description herein having reference to the figures that follow, of which:

FIG. 1 shows in diagrammatic form a breathing assistance apparatus.

FIG. 2 is a front/right side overhead perspective view of a breathing assistance apparatus with a humidifier liquid chamber positioned in the recess of the breathing assistance apparatus base unit.

FIG. 4 is a right side view of the elbow showing the location of temperature sensor(s) in broken lines.

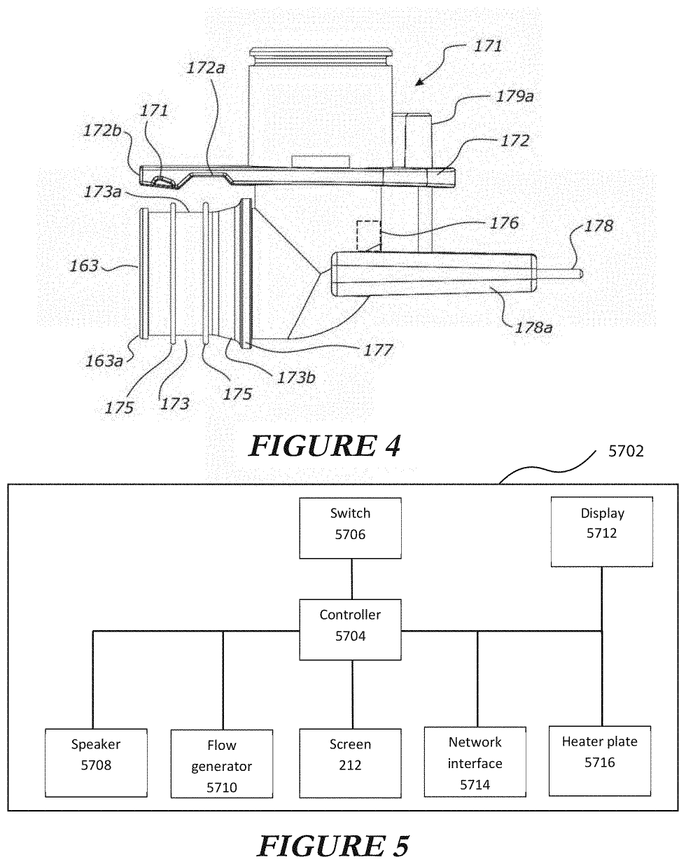

FIG. 5 is a schematic of a breathing assistance apparatus.

FIG. 6, 6A and 6b are flowcharts for the controller operation.

FIG. 7 is a flowchart of a process for determining deviations to answers of a personal health enquiry based on preset baselines.

FIG. 8 is a flowchart of a process for conditionally presenting a personal health enquiry to a patient of a breathing assistance apparatus.

FIGS. 9-18 show a user interface presenting a personal health enquiry on a breathing assistance apparatus.

FIG. 19 shows a patient and device management platform based dashboard presenting a plot formed based a plurality of answers to a plurality of queries of a personal health enquiry.

FIG. 20 shows the plot of FIG. 19 tracking the answers against time based on a plurality of symptom criteria and a plurality of medication criteria.

FIG. 21 shows answers against time based on a plurality of symptom criteria and a plurality of medication criteria.

FIG. 22 shows an deterioration overview for a healthcare provider

FIG. 23 shows an efficacy overview.

FIG. 24 is a flow diagram showing a system for providing breathing assistance or providing respiratory therapy to a patient.

### DETAILED DESCRIPTION

Patients who suffer from COPD, or bronchiectasis, or other respiratory distress are often treated with various therapies such as for example bilevel pressure therapy and/or nasal high flow therapy in a hospital. Nasal high flow therapy is one commonly used therapy for treating COPD or bronchiectasis patients in a hospital. When these patients are discharged from the hospital, his or her doctor (i.e. physician) may wish for them to continue with regular high flow therapy at home, including a retirement village or a hospice, or a location outside of hospital. In such cases, his or her doctor will prescribe in-home high flow therapy and his or her healthcare provider will provide them with a nasal high flow device suited to in-home use. Alternatively, the healthcare provider may prescribe a therapy for use at home, for example nasal high flow at home. The patient may contact an equipment provider may provide a medical device to the patient based on the prescription.

The prescription may be a flow rate that is set by a clinician for a specific patient.

The prescription may be a combination of flow rate and/or O2 concentration (i.e. O2%) and/or humidity level (e.g. dew point or RH or absolute humidity). The prescription may be loaded and stored in the memory of the apparatus, or may be stored in an .

Prescription is preferably determined by a clinician prior to discharge from the hospital or during regular check ups. The prescription may be updated by the clinician.

As used herein the term “healthcare provider” includes any party that provides healthcare, such as a hospital system, physician, medical consultant, or any other healthcare professional.

Healthcare providers often feel that it is prudent (for example financially and/or clinically) to cover the costs of providing discharged COPD patients with in-home nasal high flow devices, as in-home nasal high flow treatment may provide respiratory support to a patient therefore not requiring the patient to remain in hospital and/or reduce the likelihood of exacerbations and, and, therefore, expensive hospital readmissions.

Unfortunately, some patients that are treated with in-home therapy e.g. nasal-high flow therapy may still experience a COPD exacerbation (i.e. i.e. a flare up or worsening of COPD symptoms), or a deterioration of other health conditions that can lead to hospitalization. As such, an ability to predict whether a patient's medical state is going to deteriorate to the point of hospitalization (for example by monitoring a patient's condition) would be beneficial for both patients and healthcare providers: doctors would be able to step in and treat the patients before they suffer an exacerbation (for example a worsening of symptoms) of COPD that would require hospitalization, or, more generally, before they reach a condition that requires expensive hospitalization.

The patient's condition may be monitored over a period of time and then based on the monitoring a prediction may be made as to whether a patient's medical state is going to deteriorate to the point of hospitalization (for example before an exacerbation).

One way to gather the necessary information—for predicting a medical state which requires hospitalization—is to have patients regularly answer queries about his or her current state of health. However, this is problematic because patients, who are receiving in-home treatment, may be too unwell or may be quite old or weak to regularly fill out questionnaires on paper or with an additional device (e.g., mobile phone, tablet, desktop, laptop). Furthermore, even if patients are physically well enough, they may self-diagnose or this task may become so tedious that they disengage and stop doing it—especially if the enquiry is not user friendly (e.g., a paper form or a poorly laid out digital enquiry). Using paper or an additional user device (e.g. a mobile phone, tablet, desktop, laptop) to complete the questionnaire can be tedious for a patient because the patient has to use another device and hence patient's often don't complete questionnaires provided on paper or other devices.

In some embodiments, each time a patient activates the breathing assistance apparatus, the apparatus can present a series of queries on one or more touch screens.

The queries form part of the enquiry (i.e. enquiry form). The breathing assistance apparatus records the patient's answers to the queries. In one embodiment the query pertains to a “health parameter”. In some embodiments the query relates to non-health related parameters.

As used herein, the term “health parameter” refers to a measureable factor that relates to a patient's mental and/or physical condition. In some embodiments the health parameter can be a subjective factor related to a patient's perception of their health. For example, this may be a patient's general feeling.

In some embodiments, the health parameter may be based on a patient parameter (for example a parameter i.e. respiratory rate, oxygen saturation of the patient measured by the apparatus as described in more detail below.

The health parameter may be a subjective qualitative factor based on a patient's perception of their health such as throat soreness, degree of breathing difficulty and severity and/or type and/or frequency of coughing. The health parameter may be an objective quantitative factor that is directly measureable such as sputum colour (based on a colour chart), medicine use (e.g. frequency and amount of medicine such as for example antibiotic, steroid or inhaler).

The health parameter may be determined based on the answer to a single query, or based on the answers at least two queries.

At least some of the health parameters are indicative of COPD symptoms that a COPD patient can often suffer. COPD is chronic obstructive pulmonary disease. COPD is a respiratory disease characterised by inflammation of the airways. At least some of the health parameters may be indicative of other obstructive pulmonary diseases. At least some of the health parameters may also be indicative of dyspnea and/or respiratory distress, and/or bronchiectasis.

The breathing assistance apparatus may comprise one or more touch screens which display after the apparatus is turned on (e.g. booting). In some embodiments the, or each, touch screen displays a query. Alternately, the query may be implemented as multiple pages that can be scrolled through. Alternately, the query may be presented as a single document that is scrolled through to respond to the queries. Alternately each screen displays a separate query, and each time the patient answers a query, the next query is presented on the screen.

In some embodiments the queries are displayed on an user I/O interface (as described in more detail below).

In some embodiments, the breathing assistance apparatus may comprise an internal clock, the internal clock may maintain the current date and time. The internal clock may be part of the controller of the breathing assistance apparatus, and/or part of the patient and device management platform.

The query may be presented based on the current date and time. For example the query may be presented once daily based on information from the internal clock.

In one example implementation the query is presented once when the device is switched ON.

The query may be presented at an initial use each day (for example each day of the week), or each predetermined time period.

In some embodiments the internal clock is a real-time clock (RTC).

In some embodiments, after the patient answers the last query of the enquiry, the breathing assistance apparatus displays a therapy control screen, which the patient may use to initiate therapy.

In some embodiments, the patient may skip the enquiry if, for example, they are feeling too unwell to complete it. If the patient skips the enquiry, the device will enable therapy to begin without the need for the patient to answer the enquiry. For example, the touch screen will display a therapy control screen, which the patient may use to initiate therapy.

The therapy control screen may present multiple selectable operative parameters of the breathing assistance apparatus e.g. a flow rate or a temperature of gases provided to the patient (i.e. a patient end temperature), an 02 concentration or other such operative parameters.

The patient may be required to complete the enquiry (for example comprising a number of queries) and respond to each query in the enquiry presented on the screen of the breathing assistance apparatus. The patient may be able to skip the enquiry if the patient has completed the enquiry at least once per day.

In some embodiments, the controller may keep track of the answers to the queries from the patient. After all the queries of the enquiry are answered, the controller may determine the enquiry to be complete.

As described above after the patient answers the last query of the enquiry the controller may determine the enquiry complete.

In some embodiments the enquiry is presented on the touch screen while the breathing assistance apparatus is setting up.

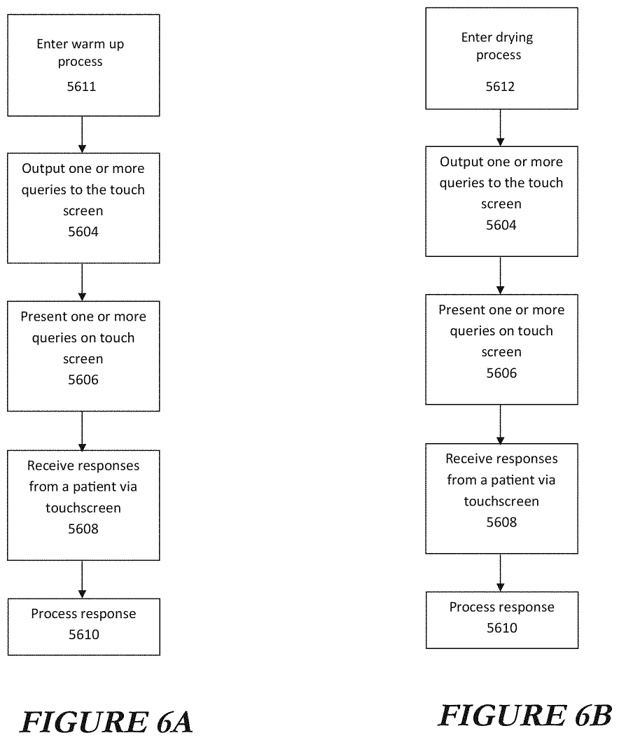

In some embodiments, for example as shown in FIG. 6A the enquiry is presented to the user for example on the touch screen during a warm up process 5611.

The warm up process may enable the humidity in the breathing circuit to reach the desired level as quickly as possible while minimising condensation and/or thermal overshoot.

The warm-up process comprises activating the heater plate of the breathing assistance apparatus to warm up the contents of the humidification chamber.

The warm up process may comprise warming the heater plate of the breathing assistance apparatus to specific temperature. For example the specific temperature may be a predetermined temperature (for example a standby temperature). The predetermined temperature may be less that an operational temperature of the heater plate when the humidifier is providing humidification. The specific temperature may be about 35 degrees Celsius.

Controlling the heater plate comprises controlling a power provided to the heater plate.

The apparatus heater plate may comprise a heater plate temperature sensor, and the controller may control the heater plate temperature to the specific temperature of the warm up process for example via for example closed loop control.

In some embodiments, the warm up process may comprise warming the heater plate of the breathing assistance apparatus to achieve a specific temperature.

In some embodiments, the warm up process comprises warming the heater plate of the breathing assistance apparatus to control the chamber outlet temperature to a specific temperature.

The chamber outlet as described elsewhere is the outlet of a humidification chamber, and optionally measured in an elbow located after the chamber outlet of the humidification chamber.

The specific temperature may be based on one or more temperature set points (as one or more operative parameters of the apparatus for therapy) of the apparatus. The set points may be entered by the user via the therapy control screen as described elsewhere, for example a desired chamber outlet temperature, a desired dew point temperature (chamber outlet or at the end of the breathing conduit), or a desired patient end temperature (at the end of the breathing conduit.)

The specific temperature may be within about 5 degrees Celsius to about 15 degrees Celsius, or about 10 degrees Celsius, or optionally about 5 degrees Celsius to about 15 degrees Celsius, or about 10 degrees Celsius less than one or more temperature set points.

One or more temperature set points may correspond to a required relative humidity or a required absolute humidity, optionally the relative humidity is about 90% to about 100% or is about 100%.

In some embodiments, the warm up process may comprise warming the heater plate of the breathing assistance apparatus to achieve a humidity of the gases (for example at the chamber outlet or at the end of the breathing conduit).

The warm up process may additionally or alternatively comprise controlling the heater wire 16a in the conduit attached to the apparatus configured to provide gases to a patient, to control a temperature at the end of the conduit to an end of the conduit specific temperature.

The end of the conduit specific temperature may be based on one or more temperature set points (optionally as one or more operative parameters of the apparatus for therapy) of the apparatus (for example as described above).

The end of the conduit specific temperature may be determined by the controller based on the selected one or more temperature set points that represents a desired humidity.

The end of the conduit specific temperature predetermined temperature (for example 35 degrees Celsius).

The one or more temperature set points may be a desired patient end temperature.

The end of the conduit specific temperature may be within about 2 degrees Celsius to about 5 degrees Celsius, or about 2.5 degrees Celsius of the desired patient end temperature, and optionally, about 2 degrees Celsius to about 5 degrees Celsius, or about 2.5 degrees Celsius less than the desired patient end temperature.

The warm up process may be complete once the specific temperature has been reached.

The apparatus can provide an audible or visual indication e.g. a visual indicia once the warm up process is completed.

In some embodiments the warm up process may comprise running the flow generator (for example a blower) at a predetermined flow rate or a predetermined flow generator output (for example a predetermined motor speed). During the warm-up process no therapeutic flow is being provided to the patient.

The predetermined flow rate may be a low flow rate sufficient to ensure that high temperature and/or high humidity gases do not collect in the humidification chamber.

The predetermined flow rate may be lower than a therapeutic flow being provided to the patient. The predetermined flow generator output may be a predetermined motor speed may be about 1000 RPM to about 3000 RPM or less than about 2000 RPM.

The heater plate may be provided with an activation signal by the controller to warm up the heater plate. The enquiry and the queries comprising the enquiry are presented on the touchscreen (or other user I/O interface) of the breathing assistance apparatus during the warm up process (for example as shown in FIG. 6A). This advantageous because the patient is often waiting until the warm up process is completed.

The warm up process can be several minutes e.g. between 5 mins to 25 mins. This provides a suitable time period where flow therapy is not being used by the patient, thereby allowing the patient free time to respond to the enquiry (i.e. respond to the queries presented on the touch screen).

Presenting the queries to the user during the warm-up process allows the patient to use the time where usually they would be waiting for the machine to warm up to answer the queries. Utilising the warm up process to present the queries may increase the likelihood of the patient answering the queries. This is because the patient is engaged and using the breathing assistance apparatus.

In some embodiments the therapy control screen may be presented on start-up of the device.

In some embodiments the warm up process may be activated on start-up of the device.

In some embodiments, the warm up process may be activated when the user is prompted to enter one or more operative parameters of the breathing assistance apparatus (optionally via a therapy control screen).

In some embodiments, the warm up process may be activated when the user begins to enter one or more operative parameters of the breathing assistance apparatus (optionally via a therapy control screen).

In some embodiments, the warm up process may be manually activated by a user.

In some embodiments, the warm up process is activated once the user has initiated therapy (for example via the therapy control screen).

In some embodiments, the breathing assistance apparatus displays a therapy control screen on start-up, which the patient may use to initiate therapy (as described above). Once the user has entered one or more operative parameters of the breathing assistance apparatus on the therapy control screen (e.g. a flow rate or a temperature of gases provided to the patient (i.e. a patient end temperature), an 02 concentration or other such operative parameters) the apparatus may enter the warm up process and optionally display the enquiry (for example as described above).

In some embodiments, for example as shown in FIG. 6B the enquiry is presented to the user for example on the touch screen during a drying process 5612.

The drying process may be configured to evaporate remaining condensate in the apparatus and/or patient breathing conduit and/or patient interface.

The drying process may be as described in WO2006126900. The contents of that specification are incorporated herein in its entirety by way of reference.

The drying process may comprise activating the heater wire 16a in the patient breathing conduit 16 while the flow generator provides gases as a set flow rate or predetermined motor speed. The set flow rate may be lower than a therapeutic flow being provided to the patient. The predetermined motor speed may be about 1000 RPM to about 3000 RPM or less than about 2000 RPM.

Additionally, or alternatively, the drying process may comprise controlling the heater plate of the humidifier to a predetermined value. The predetermined value may be low enough to prevent the generation of humidity by the humidifier. Alternatively, the heater plate may be deactivated during the drying process.

The heater wire 16a may be controlled to a predetermined temperature at for example the end of the patient breathing conduit, or controlled to a predetermined duty cycle or to a predetermined power.

The predetermined duty cycle may be 100%.

The predetermined temperature may be greater than 45 degrees Celsius.

The drying process may be configured to operate for at about 20 minutes to about 40 minutes, or about 15 minutes.

During the drying process, the flow generator may be set to a predetermined flow rate optionally sufficient to ensure a flow of gas through the patient breathing circuit to remove any evaporated condensate. The predetermined flow rate may be about 10 litres/minute to about 20 litres/minute.

The drying process may be undertaken at the end of a therapy session. The patient (or other healthcare professional) may indicate when the therapy session has ended, or in some embodiments the therapy session may have one or more conditions which are met to signal an end to the therapy session (for example a time elapsed).

The drying process may be manually activated by the patient or healthcare provider.

In some embodiments the end of therapy may be determined by the detection that the user has taken off the patient interface. The apparatus may detect that the user has taken off the patient interface based on a flow signal measured from the flow sensor. Optionally, the end of therapy may be determined by the detection that the user has taken off the patient interface and a predetermined period of time has elapsed.

In some embodiments, the apparatus may detect that the user has taken off the patient interface when breathing is not detected. Breathing may be detected based on a flow signal measured from the flow sensor.

In some embodiments, the drying process may be activated when a standby process has been active for a predetermined amount of time. The standby process may comprise running the flow generator (for example a blower) at a predetermined flow rate (or for example a predetermined motor speed) when no therapeutic flow is being provided to the patient. The predetermined flow rate may be lower than a therapeutic flow being provided to the patient. The predetermined motor speed may be about 1000 RPM to about 3000 RPM or less than about 2000 RPM.

The standby process may be manually activated by the patient or healthcare provider.

The point in time that the drying mode is deactivated may be determined by a number of methods. The first of which is to measure the temperature of gases at the chamber outlet (for example at the elbow 30/171). When the temperature of the gases at the chamber outlet drops below or equals ambient temperature (which is preferably measured by an additional temperature sensor, for example, located at the flow generator or elsewhere in the housing of the apparatus) the flow generator and heater wire 16a are powered off by the controller. In this way gases flow through the humidification chamber and as the heater plate cools the humidity of the gases flowing through the conduit reduces and the conduit 3 becomes drier.

A second method for determining when the drying mode is to be deactivated by the controller is to turn off the power to the humidification chamber and maintain a gases flow through the conduit at a fixed speed or pulses and maintain power in the heater wire 16a to evaporate any condensate off the walls of the conduit. After a predetermined time, preferably in excess of one minute, the heater wire 16a and flow generator) would be switched off. Yet another method of controlling the drying of the conduit is to switch the flow generator) off and switch off the power to the heater plate power. The controller would then compare the heater plate temperature with an ambient temperature that is measured either inside or outside the apparatus (as previously described). When this temperature difference or comparison is within a predetermined limit, which typically approximates zero, a flow of gases is caused to flow in the same manner as described above.

It is possible that it could take longer to dry the condensate in the conduit than to cool the chamber. In this instance it may be necessary to extend the drying mode for some time, for example, up to 30 minutes depending on the ambient temperature of the water remaining in the chamber.

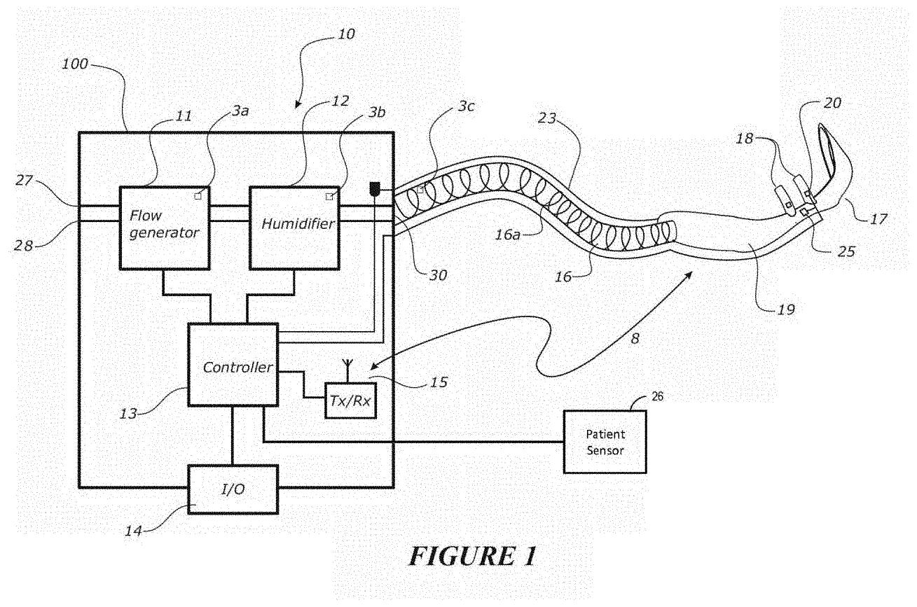

A breathing assistance apparatus as a high flow apparatus for delivering a flow of gas (which may contain one or more gases) to a patient is shown in FIG. 1.

Alternatively, the apparatus could, for example, be a CPAP apparatus and/or a Bi-level device (or any combination thereof). The apparatus could, for example, provide CPAP therapy and/or Bi-level pressure therapy (or any combination thereof).

Different patient interfaces may be provided depending on the therapy type. For example a non-sealing interface may be provided for high flow therapy, and a sealing interface may be provided

An exemplary apparatus is described in WO 2011/056080. The contents of that specification are incorporated herein in its entirety by way of reference.

A breathing assistance apparatus comprises a gases supply and optionally gases humidification apparatus. The breathing assistance apparatus is operable to provide respiratory assistance to patients or users who require a supply of gas (humidified or otherwise) at positive pressure for the treatment of diseases such as Obstructive Sleep Apnea (OSA), snoring, or Chronic Obstructive Pulmonary Disease (COPD) and the like. A breathing assistance apparatus would typically include a humidifier chamber as a humidifier liquid chamber, so as to form a combined assisted breathing unit and humidifier.

Breathing assistance apparatuses, when used with a humidifier, typically have a structure where gases at a required pressure are delivered from an assisted breathing unit or blower unit to a liquid chamber downstream from the blower. As the gases pass through the liquid chamber, they become saturated with liquid vapour (e.g. water vapour). A flexible tubular gases conduit delivers the gases to a user or patient downstream from the humidifier chamber.

A high flow apparatus may be used to deliver a high gas flow or high flow therapy to a patient to assist with breathing and/or treat breathing disorders including chronic obstructive pulmonary disease (COPD) or respiratory distress syndrome or dyspnea, or bronchiectasis. A high flow apparatus includes a gases supply and typically includes a humidification apparatus. A high flow apparatus may provide respiratory support to a patient.

A CPAP apparatus may be used to deliver a continuous positive airway pressure to patient, or CPAP therapy to a patient (as described in more detail below).

A Bi-level apparatus may be used to deliver a bi-level pressure to the patient, or Bi-level therapy to a patient (as described in more detail below).

The breathing assistance apparatuses typically have one or more accessories such as a breathing conduit and a patient interface such as a cannula or mask for delivering gases to a patient. The conduit enables gases to be delivered from the housing of the breathing assistance apparatus to the patient. For example, the apparatus may be placed on a floor or other support surface, and the patient may be in a bed.

The breathing assistance apparatus may have a recess for receipt of a humidifier liquid chamber. The liquid chamber will receive liquid from, for example, a flexible liquid bag that delivers liquid to a humidifier liquid chamber via one more tubes. Alternatively, the liquid chamber can be removed and refilled as required. The recess will contain a heater plate to heat the liquid chamber, to humidify gases passing through the liquid chamber. The humidified gases are then delivered to the patient.

FIG. 1 shows an example breathing assistance apparatus 10. The breathing assistance apparatus 10 is configured to provide high flow therapy and function as a high flow apparatus.

The breathing assistance apparatus 10 is further configured to provide pressure therapy and can function as a pressure therapy apparatus.

The breathing assistance apparatus 10 may be configured to provide high flow of gases when in a high flow mode. The breathing assistance apparatus 10 may be configured to operate in a pressure therapy mode, where the breathing assistance apparatus 10 provides a pressure therapy. The pressure therapy may be a positive bi-level pressure therapy or a constant positive pressure therapy. For example in bi-level pressure therapy the apparatus 10 is configured to control the gases flow such that the patient receives an inspiratory pressure and an expiratory pressure, wherein both pressures are positive pressures. In constant pressure therapy the apparatus 10 is configured to deliver gases at a constant pressure that may be user set. The level or pressure may be set by a healthcare provider (e.g. a physician). An unsealed patient interface is used to deliver high flow therapy e.g. a nasal cannula or a tracheal interface. Conversely a sealed patient interface is used to deliver a pressure therapy (e.g. a nasal mask or a full face mask or nasal pillows).

In general terms, the apparatus 10 comprises a main housing 100 that contains a flow generator 11, a humidifier 12, a controller 13, and a user I/O interface 14 (comprising, for example, a display and input device(s) such as button(s), a touch screen, or the like).

In some embodiments the user I/O interface may be part of an ancillary device. The ancillary device may be for example a phone, tablet, or computer. The ancillary device may be configured to communicate directly with the apparatus, or be configured to communicate with the apparatus via one or more servers.

In some embodiments, the queries of the enquiry may be presented on the user I/O interface of the ancillary device, and the user may answer the queries on the ancillary device.

In some embodiments, the answers to the queries may be stored on the ancillary device and transmitted to the apparatus (for example at the end of the enquiry, or as each query is answered, or at a later time). In some embodiment the apparatus may then upload the answers to the queries (along with other information, for example patient parameters) to the patient and device management platform.

The ancillary device may upload the answers to the queries (optionally along with other information, for example patient parameters, from the apparatus) to the patient and device management platform.

In some embodiments the touchscreen may be provided as, or as part of the user I/O interface 14

In some embodiments the controller may be comprised of a plurality of controllers to control different components of the apparatus 10.

The plurality of controllers may comprise one or more of: a controller for the user I/O interface, a controller to control the flow generator and/or the humidifier, a controller to receive sensor inputs.

In some embodiments the controller to control the flow generator and humidifier is configured to receive input from the other controllers (for example the controller to receive sensor inputs, and user controller for the user I/O interface.)

The plurality of controllers may comprise a plurality of controller to receive sensor inputs, each controller to receive sensor inputs being associated with a particular sensor, or set of sensors.

The plurality of controllers may be configured to communicate with each other (for example via a bus) and/or communicate to a master controller.

The flow generator 11 may comprise a motor/impeller arrangement e.g. a blower or pump or may comprise a compressor or other suitable component to create a flow of gases.

The controller 13 is configured or programmed to control the components of the apparatus, including: operating the flow generator 11 to create a flow of gas (gas flow) for delivery to a patient, operating the humidifier 12 to humidify and/or heat the generated gas flow, receive user input from the user interface 14 for reconfiguration and/or user-defined operation of the apparatus 10, and output information (for example on the display) to the user via the user interface 14.

In some embodiments the controller is configured to receive a start up input/request via the user interface. The startup input/request activates the apparatus to initiate the enquiry to the user.

The user could be a patient, healthcare provider, or anyone else interested in using the apparatus. In one example where the apparatus is used in an out of hospital setting e.g. in the home or in a hospice or retirement village or other non-hospital setting, the user of the apparatus 10 is the patient. The patient will use the device to receive high flow therapy or pressure therapy according to a prescription from a healthcare provider (e.g. a physician).

It will be appreciated that in the context of answering the queries (as described elsewhere) the user will preferably be the patient as the queries relate to the health parameters of the patient.

A patient breathing conduit 16 is connected to a gas flow output or patient outlet port 30 (i.e. outlet port) in the housing 100 of the breathing assistance apparatus 10, and is connected to a patient interface 17 such as a nasal cannula with a manifold 19 and nasal prongs 18. In some embodiments, the nasal cannula may be sealed or un-sealed (for example when used to provide high flow therapy).

Additionally, or alternatively, the patient breathing conduit 16 could be connected to a face mask (for example a sealed mask when pressure therapy, such as CPAP or Bi-level pressure therapy, is provided).

Additionally, or alternatively, the patient breathing conduit could be connected to a nasal pillows mask, and/or a nasal mask, and/or a tracheostomy interface, or any other suitable type of patient interface.

The gas flow, which may be humidified, that is generated by the breathing assistance apparatus 10 is delivered to the patient via the patient breathing conduit 16 (and optionally via a humidifier) through the patient interface 17. The patient breathing conduit 16 can have a heater wire 16a to heat gas flow passing through to the patient. The heater wire 16a is controlled by the controller 13. Alternatively the breathing assistance apparatus comprises a separate heater wire controller (for example as one of the plurality of controller described above) that controls an output (for example power or current or voltage) to the heater wire

The patient breathing conduit 16 and/or patient interface 17 can be considered part of the breathing assistance apparatus 10, or alternatively peripheral to it. The breathing assistance apparatus 10, breathing conduit 16, and patient interface 17 may together form a breathing assistance system, for example a flow therapy system for providing high flow respiratory support i.e. high flow respiratory therapy to a patient as illustrated in FIG. 1.

The controller 13 controls the flow generator 11 to generate a gas flow of the desired flow rate.

In some embodiments the controller controls one or more valves to control the mix of air and oxygen or other alternative gas.

In some embodiments the controller controls the humidifier 12 to humidify the gas flow and/or heat the gas flow to an appropriate level.

The gas flow is directed out through the patient breathing conduit 16 and patient interface 17 to the patient. The controller 13 can also control a heating element in the humidifier 12 and/or the heating element 16a in the patient breathing conduit 16 to humidify and/or heat the gas to a desired temperature that achieves a desired level of therapy and/or comfort for the patient.

The controller 13 can be programmed with, or can determine, a suitable target temperature of the gas flow.

The controller 13 controls the flow generator to generate a gases flow at a desired flow rate based on feedback from a flow sensor when in a high flow therapy mode. Alternatively the controller 13 is configured to control the flow generator to generate a gases flow at a desired pressure based on feedback from a pressure sensor e.g. a differential pressure sensor when in pressure therapy mode.

Operation sensors 3a, 3b, 3c, 20, and 25, such as flow, temperature, humidity, and/or pressure sensors, can be placed in various locations in the breathing assistance apparatus 10 and/or the patient breathing conduit 16 and/or patient interface 17.

In some embodiments, at least one of the operation sensors 3a, 3b, 3c, 20, and 25 is provided within a sensor module. The sensor module is be located in the gases flow path. The sensor module may be be located in the gases flow path between the flow generator (for example the blower) and the humidifier.

At least one of the operation sensors 3a, 3b, 3c, 20, and 25 may be provided within the gases flow path to sense a parameter of the gases flow.

In some embodiments, the apparatus may measure one or more patient parameters. The patient parameters may be for example one or more of: respiratory rate, oxygen saturation of the patient. The patient parameters may be or relate to one or more physiological parameters of the patient. The patient parameter may be measured by the patient sensor (as described below) or determined based on sensors from the apparatus).

In some embodiments, the respiratory rate may be determined based on a flow signal from a flow sensor. The respiratory rate may be for example determined as disclosed in WO2019/102384. The contents of that specification are incorporated herein in his or her entirety by way of reference.

Also included is a patient sensor 26. The patient sensor 26 may be a sensor that is mounted on the patient or associated with the patient to measure the patient parameter. In one example the patient sensor 26 is pulse oximeter that measures the oxygen saturation of the patient i.e. SpO2 value. Output from the sensors can be received by the controller 13, to assist it to operate the breathing assistance apparatus 10 in a manner that provides optimal therapy.

In some configurations, providing optimal therapy includes meeting, or exceeding, a patient's inspiratory flow. The apparatus 10 may have a transmitter and/or receiver 15 (for example as part of the network interface described in more detail below) to enable the controller 13 to receive signals 8 from the sensors and/or to control the various components of the breathing assistance apparatus 10, including but not limited to the flow generator 11, humidifier 12, and heater wire 16a, or accessories or peripherals associated with the breathing assistance apparatus 10.

Additionally, or alternatively, the transmitter and/or receiver 15 may deliver data to an external or remote service or platform. For example, a remote patient and device management platform. The patient and device management platform may be any one or a combination of a remote device, server, an application, a cloud service (for example distributed computer system resources) or any other suitable hardware and software platform. In one embodiment the transmitter and/or receiver 15 may enable remote control of the apparatus 10.

The breathing assistance apparatus 10 may be any suitable type of apparatus, but in some configurations may deliver a high gas flow or high flow therapy (of e.g. air, oxygen, other gas mixture, or some combination thereof) to a patient to assist with breathing and/or treat breathing disorders. In some configurations, the gas is or comprises oxygen. In some configurations, the gas comprises a blend of oxygen and ambient air. High flow therapy as discussed herein is intended to be given its typical ordinary meaning as understood by a person of skill in the art which generally refers to a respiratory assistance system delivering a targeted flow of respiratory gases (preferably humidified gas) via an intentionally unsealed patient interface with flow rates generally intended to meet or exceed inspiratory flow of a patient. Typical patient interfaces include, but are not limited to, a nasal or tracheal patient interface. Typical flow rates for adults often range from, but are not limited to, about fifteen liters per minute (LPM) to about seventy liters per minute or greater. Typical flow rates for pediatric patients (such as neonates, infants and children) often range from, but are not limited to, about one liter per minute per kilogram of patient weight to about three liters per minute per kilogram of patient weight or greater. High flow therapy can also optionally include gas mixture compositions including supplemental oxygen and/or administration of therapeutic medicaments. High flow therapy is often referred to as nasal high flow (NHF), humidified high flow nasal cannula (HHFNC), high flow nasal oxygen (HFNO), high flow therapy (HFT), or tracheal high flow (THF), among other 10 common names.

For example, in some configurations, for an adult patient ‘high flow therapy’ may refer to the delivery of gases to a patient at a flow rate of greater than or equal to about 10 liters per minute (10 LPM), such as between about 10 LPM and about 100 LPM, or between about 15 LPM and about 95 LPM, or between about 20 LPM and about 90 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 50 LPM and about 60 LPM. In some configurations, for a neonatal, infant, or child patient ‘high flow therapy’ may refer to the delivery of gases to a patient at a flow rate of greater than 1 LPM, such as between about 1 LPM and about 25 LPM, or between about 2 LPM and about 25 LPM, or between about 2 LPM and about 5 LPM, or between about 5 LPM and about 25 LPM, or between about 5 LPM and about 10 LPM, or between about 10 LPM and about 25 LPM, or between about 10 LPM and about 20 LPM, or between about 10 LPM and 15 LPM, or between about 20 LPM and 25 LPM. A high flow therapy apparatus with an adult patient, a neonatal, infant, or child patient, may, in some configurations, deliver gases to the patient at a flow rate of between about 1 LPM and about 100 LPM, or at a flow rate in any of the sub-ranges outlined above. Gases delivered may comprise a percentage of oxygen. In some configurations, the percentage of oxygen in the gases delivered may be between about 20% and about 100%, or between about 30% and about 100%, or between about 40% and about 100%, or between about 50% and about 100%, or between about 60% and about 100%, or between about 70% and about 100%, or between about 80% and about 100%, or between about 90% and about 100%, or about 100%, or 100%.

High flow therapy has been found effective in meeting or exceeding the patient's inspiratory flow, increasing oxygenation of the patient and/or reducing the work of breathing. Additionally, high flow therapy may generate a flushing effect in the nasopharynx such that the anatomical dead space of the upper airways is flushed by the high incoming gas flows. This creates a reservoir of fresh gas available for each and every breath, while reducing re-breathing of carbon dioxide, nitrogen, etc.

In one example for high flow therapy, an unsealed or non-sealing user interface, e.g. a nasal cannula, is used. For CPAP or other pressure therapy a sealed interface is typically used, e.g. a nasal mask, full face mask, or nasal pillows.

The patient interface 17 may be a non-sealing interface to prevent barotrauma when the apparatus is providing high flow therapy (e.g. tissue damage to the lungs or other organs of the respiratory system due to difference in pressure relative to the atmosphere). The patient interface may be a nasal cannula with a manifold and nasal prongs, and/or a face mask, and/or a nasal pillows mask, and/or a nasal mask, and/or a tracheostomy interface, or any other suitable type of patient interface. The patient interface may comprise a headgear configured to maintain the interface on the face of the user.

As described below, the breathing assistance apparatus 10 has various features to assist with the functioning, use, and/or configuration of the breathing assistance apparatus 10.

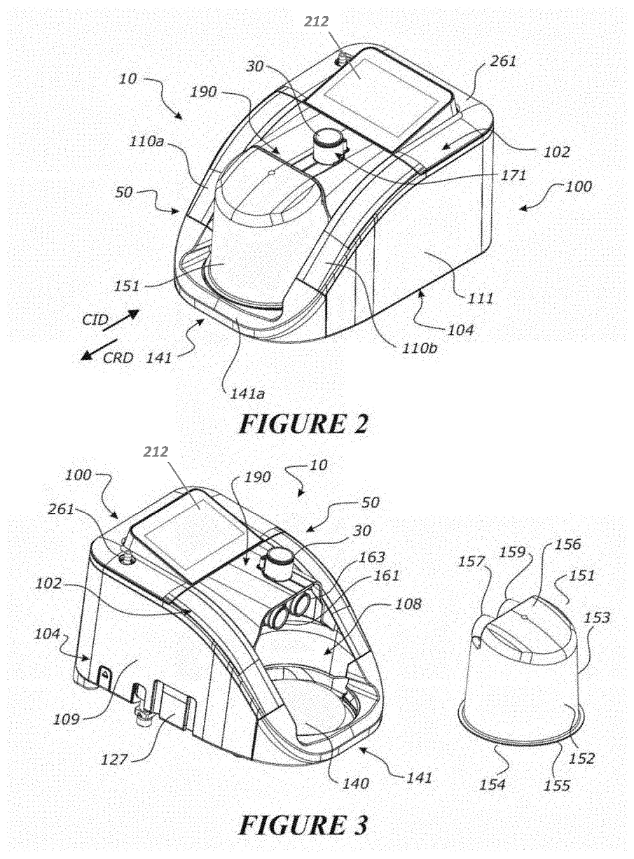

As shown in FIGS. 2 and 3, a first configuration breathing assistance apparatus 10 comprises a breathing assistance apparatus base unit 50 having a main housing 100. The main housing 100 has a main housing upper chassis 102 and a main housing lower chassis 104.

The main housing of the base unit 50 has a peripheral wall arrangement. The peripheral wall arrangement defines a recess 108 that provides a humidifier liquid chamber bay for receipt of a removable liquid chamber 151. The removable liquid chamber 151 contains a suitable liquid such as water for humidifying gases that will be delivered to a patient.

The base unit 50 of the apparatus 10 may have a movable finger guard 140 that guards against a user touching a base flange 155 of the liquid chamber when the liquid chamber is in place in the recess 108 and when a barrier 141a of the finger guard is in a covering position as shown in FIG. 2. The barrier 141a is movable between the covering position and a lowered access position in which the recess 108 is less covered or is uncovered by the barrier 141a.

In the form shown, the main housing lower chassis 104 peripheral wall arrangement comprises a substantially vertical left side outer wall 109 that is oriented in a front-to-rear direction of the main housing 100, a substantially vertical right side outer wall 111, and a substantially vertical rear outer wall that extends between and connects the walls 109, 111. A bottom wall 115 extends between and connects the lower ends of walls 109, 111, 113, and forms a base of the apparatus and a substantially horizontal floor portion of the liquid chamber bay.

The floor portion of the recess 108 has a receptacle portion 108a to receive a heater arrangement such as a heater plate 140 or other suitable heating element(s) for heating liquid in the liquid chamber 151 for use during a humidification process. The heater plate would typically have a shape that substantially corresponds to the shape of a base 154 of the liquid chamber 151, such as a circular shape for example. The heater plate 140 is resiliently mounted; for example, on biasing device(s) such as spring(s). The resilient mounting enables the heater plate to move downwardly to accommodate the liquid chamber 151 in the recess 108, while maintaining good contact between the heater plate 140 and the base of the liquid chamber once the liquid chamber is inserted in the recess 108.

The main housing lower chassis 104 is attachable to the upper chassis 102, either by suitable fasteners or integrated attachment features such as clips for example. When the main housing lower chassis 104 is attached to the main housing upper chassis 102, the walls of the upper and lower chassis engage with each other.

The lower chassis 104 has a motor recess for receipt of a motor module which may be permanently inserted in the recess or may be removable from the recess. A recess opening is provided in the bottom wall 115 adjacent a rear edge thereof, for receipt of the removable motor module. A base 123 of the motor module covers the opening into the motor recess 121. The base may be fixed after assembling the base to lock the motor module within the motor recess to prevent tampering with the motor. The motor module comprises a motor that forms a blower to cause gas flow, and may comprise one or more sensors to sense properties of the gas passing through the motor module. The motor module may comprise sensor(s) to sense parameters of gases flowing through the motor module. In one example the motor module may comprise a sensing module that supports a plurality of sensors e.g. a flow sensor, a differential pressure sensor, a gas composition sensor, a humidity sensor and/or any other sensors. The sensors are arranged to be in electronic communication with the controller such that the controller can receive sensor outputs to be used by the controller during control of the apparatus and its components.

The motor module and housing of the base unit 50 of the apparatus 10 are provided with suitable tubes and/or gas flow passages to deliver gases from one or more gases inlets of the base unit 50 of the apparatus, to a gas inlet port 157 of the liquid chamber 151 to humidify the gases. The gases are delivered from a gas outlet port 159 of the liquid chamber 151 to the patient outlet port 30 (via a humidified gas inlet port 163) and thereby to the patient via the patient breathing conduit 16 and patient interface 17.

The housing may comprise two gases inlets 27, 28. The first inlet may be an ambient air inlet and the second inlet may be for a supplementary gas e.g. oxygen or heliox or another supplementary gas. In the illustrated example the supplementary gas is oxygen. The supplementary gas source may comprise a valve that is controlled by the controller 13 to regulate the amount of supplementary gases introduced into the apparatus 10. The air and supplementary gases are mixed by the flow generator i.e. the blower.

The motor recess 122 comprises a recess opening in a bottom wall 115 of the housing. Alternatively, the recess opening could be in a different part of the housing, such as a side, front, or top of the housing.

The base unit 50 of the apparatus 10 may have a battery module 125 to provide power to the apparatus when there is a power outage or for portable use. The battery module comprises a battery cover 126 containing a battery. The battery of the battery module 125 may be replaceable.

The battery module 125 may provide power if mains power is disconnected. In some embodiments the controller is configured to detect disconnection of mains power has and automatically switch to draw power from the battery module 125 to provide functions of the breathing assistance apparatus.

When the battery module 125 is utilized to power the apparatus, the apparatus may operate for a specific amount of time e.g. 30 mins to 1 hour.

In the form shown, the battery cover 126 of the battery module 125 is coupled to an exterior of the back wall 113 of the apparatus housing 100. This provides a large surface area to cool the battery and reduces the amount of heat entering the apparatus from the battery. Additionally, this configuration reduces the influence of heat generated by components of the apparatus on the battery, particularly when the battery is being charged. In an alternative configuration, the battery may be internally mounted in the main housing.

The housing may be provided with a battery cover 126 to cover the battery once installed. Alternatively, the battery may mount directly to the housing 100 without a cover. The battery, and therefore the battery cover 126, may be sized to not extend beyond the bottom wall 115 of the housing. Alternatively, the battery cover 126 may be longer and extend beyond the bottom wall 115 of the housing to accommodate a larger battery.

As shown in FIG. 3, the base unit 50 of the apparatus 10 has a mounting feature 127 for mounting the apparatus to a support apparatus.

The mounting feature 127 may be integrally formed with part of the main housing of the base unit 50 of the apparatus 10. In the form shown, the mounting feature 127 is integrally formed with the left side wall 109 the lower chassis 104 of the housing. The mounting feature 127 could instead be integrally formed with any of the other walls of the housing, such as a rear wall, right side wall, or other wall.

The main housing of the apparatus may be formed from any suitable material that will allow the mounting feature 127 to be integrally formed. For example, the housing may be formed from polycarbonate.

The integral mounting feature 127 has greater impact strength compared to an additional, screwed in part. Strengthening of the mounting feature 127 may also be done by, for example, varying the wall thickness, ribbing, or varying in internal geometries.

FIG. 3 shows a humidifier liquid chamber 151 (i.e. a reservoir) for use with the breathing assistance apparatus 10. The chamber 151 is a removable liquid chamber to be filled with liquid such as water for the humidification of respiratory gases. The liquid chamber 151 is removable from the base unit 50 of the breathing assistance apparatus 10 to be more easily re-filled or disposed of.

The liquid chamber 151 has a body 152 having a peripheral wall 153 20 and a roof 156. The body defines an internal chamber for receipt of a liquid. A base 154 is provided at the lower end of the peripheral wall, and comprises a base flange 155 that projects outwardly from the lower end of the peripheral wall 153. First and second base unit connection ports comprising a liquid chamber gas inlet port 157 and a liquid chamber gas outlet port 159 are in communication with the internal chamber of the liquid chamber 151. The breathing assistance apparatus base unit 50 comprises complementary chamber connection ports comprising a gas outlet port 161 and a humidified gas inlet port 163. When the liquid chamber is received in the recess 108 to engage with the housing 100, the liquid chamber gas inlet port 157 connects to the gas outlet port 161 that receives gases from the motor module via a gasflow passage, and the liquid chamber gas outlet port 157 connects to the humidified gas inlet port 163 to deliver humidified gases from the liquid chamber to the patient outlet port 30.

The liquid chamber could have a generally circular peripheral shape, or could be any other suitable shape, with the recess 108 shape modified accordingly if required.

In the form shown, the liquid chamber 151 has a substantially cylindrical shape.

The base 154 of the liquid chamber is heat conductive. In particular, the base 154 of the liquid chamber 151 is made from a highly heat conductive material, which allows heating of the liquid in the chamber when in contact with the heater plate 140 of the base unit 50 of the breathing assistance apparatus 10 during use.

The liquid chamber 151 can be fluidly coupled to the base unit 50 of apparatus 10 in a rearward insertion direction of the liquid chamber 151 into the recess 108, from a position at the front of the housing 100 in a direction toward the rear of the housing 100. The gas outlet port 161 is in fluid communication, via a fixed L shaped elbow, with a gas flow passage from the motor/impeller unit.

The humidified gas inlet port 163 is embodied in a removable component comprising removable elbow 171 that can be removably connected to the housing. The removable elbow 171 is L-shaped, and further comprises the upstanding patient outlet port 30 for coupling to the patient breathing conduit 16 to deliver gases to the patient interface 17. In different configurations, the removable component may not have an elbow shape, and could instead, for example, have aligned inlet and outlet ports.

The gas outlet port 161, humidified gas inlet port 163, and patient outlet port 30 each comprise soft seals such as wiper seals, L-seals, T seals, X-rings, or 0-rings to provide a sealed gases passageway between the apparatus 10, the liquid chamber 151, and the patient breathing conduit 16 and optionally one or more other accessories.

The gas outlet port 161 and gas inlet port 163 comprise multiple sealing elements. The sealing elements may be wiper seals, T seals, L-seals, X-rings, or 0-rings. The wiper seals may have a T-shaped cross-section. The gas outlet port 161 and the gas inlet port 163 may each comprise two, three, or more sealing elements. In one configuration, each of the gas inlet port 163 and gas outlet port 161 comprises a pair of wiper seals. In this configuration, the gas inlet port 163 has two wiper seals positioned adjacent each other on the gas inlet port 163. Similarly, the gas outlet port 161 comprises a pair of wiper seals positioned adjacent each other on the gas outlet port 161. The pair of wiper seals (or of the other types of sealing elements) on each port 161, 163 improves the seal with the corresponding base unit connection ports 157, 159 and provides improved protection against liquid ingress into the interior of the housing of the base unit 50 of the apparatus where electronics are located. When the liquid chamber 151 is coupled to the gas inlet port 163 and gas outlet port 161 of the base unit 50, one wiper seal may be positioned inside each base unit connection port 157, 159 and one wiper seal may be located outside each base unit connection port 157, 159, when the liquid chamber is assembled with the base unit 50. Alternatively, both wiper seals are positioned inside the respective base unit connection ports 157, 159 when the liquid chamber 151 is assembled onto the heater plate 140 in the recess 108. The arrangement of using two wiper seals per port 161, 163 provides redundancy for liquid ingress. Similar arrangement can be used for L-seals, X-rings, or 0-rings. The gas outlet port 161 and gas inlet port 163 of the base unit 50 are structured to have an elongate portion; i.e., a length of the ports 161, 163 is such that the wiper seals, L-seals, X-rings, or 0-rings are retained on the ports 161, 163.

The gas inlet port 157 of the liquid chamber is complementary with the gas outlet port 161 of the breathing assistance apparatus base unit 50, and the gas outlet port 159 of the liquid chamber is complementary with the humidified gas inlet port 163 if the breathing assistance apparatus base unit 50. The axes of those ports may be parallel and/or horizontal enable the liquid chamber 151 to be inserted into the recess 108 in a substantially linear movement to form gas connections between the ports.

The chamber connection ports 161, 163 are parallel cylindrical features extending from the housing of the breathing assistance apparatus base unit 50. The ports 161, 163 will typically have an equal profile, and equal length, and axes located on the same horizontal plane. The ports 161, 163 will typically terminate on the same vertical plane at his or her distal ends. The ports 161, 163 have a port separation distance or pitch, which is the horizontal distance between the centre or axis of each port 161, 163. This is substantially equal to the horizontal distance between the centres of the base unit connection ports 157, 159 of the liquid chamber.

In some embodiments, the base unit connection ports 157, 159 of the humidification chamber (for example the liquid chamber) are pneumatically connected chamber connection ports 161, 163 of the apparatus 50 when the humidification chamber is provided to the liquid chamber bay (for example as described in more detail above).

The chamber connection ports 161, 163 (which in the form shown are male connection members) of the breathing assistance apparatus base unit 50 insert into the base unit connection ports 157, 159 (which in the form shown are female connection members) of the liquid chamber in a concentric manner. The inner diameter of the base unit connection ports 157, 159 is larger than the outer diameter of the chamber connection ports 161, 163.

The liquid chamber 151 may initially be inserted into the recess 108 on an angle, and then tilted to be substantially horizontal, so that a rear part of movement of the liquid chamber 151 is substantially linear. The recess 108 may comprise one or more guide rails to assist with holding the liquid chamber in position in the recess 108.

The breathing assistance apparatus 10 may have any one or more of the features and/or functionality of the breathing assistance apparatus described and shown in WO 2016/207838. The contents of that specification are incorporated herein in his or her entirety by way of reference.

In order to prevent gas leaking from either of the two connections (port 157 to port 161, and port 159 to port 163), one or more sealing elements are provided for each connection. The one or more sealing elements may be on the outer surface of male ports, and seal against the inner surface of female ports. In one configuration, the gas inlet port 157 of the liquid chamber and the gas outlet port 159 of the liquid chamber are the female ports, and the housing ports, i.e. the gas outlet port 161 and the humidified gas inlet port 163 are the male ports. Alternatively, the ports 157, 159 of the liquid chamber may be the male ports and the ports 161, 163 of the breathing assistance apparatus base unit 50 may be the female ports.

The screen 212 (refer FIGS. 2 and 3) is located on the housing (e.g., upper side, lateral side) and is positionally fixed. However, the screen 212 can be positionally adjustable (e.g., hingedly).

The screen 212 as shown in FIGS. 2 and 3 is rectangular, but can be shaped differently (e.g., square, circular). The screen 212 is mains or battery powered. The screen 212 is located on an upper surface of the housing and is also angled to improve visibility of the screen to a patient. Further the screen 212 being located on the upper surface of the housing makes the screen and the screen contents easier to view by a patient e.g. within an in home environment.

In some embodiments the screen 212 is a colour screen, and preferably a colour touch screen (e.g., resistive, capacitive). The screen 212 is large in size which makes it easier to view content presented on the screen for patients. These patients often can be quite ill e.g. COPD patients and can often be elderly. Having a large, high resolution screen 212 helps to easily convey the inquiry and the content of the inquiry to the patient. It makes the inquiry more engaging since it is easier to view. The large touch screen 212 also improves legibility of the presented content which further helps to make the content more engaging.

The large resolution and the colour touch screen 212 allows presentation of various queries that may require colours or shades of colours to be presented (for example sputum colour). The screen 212 being a touchscreen also allows for easier interaction with the patient as the touchscreen is simpler to use and more intuitive to use since user is required to touch the screen 212 and perform gestures on the screen to input information. The touchscreen avoids the need to use buttons or dials that may require a complex sequence to be pushed to input data e.g. respond to the content of the inquiry.

The resolution of the screen 212 is sufficiently large to make the presented content easy to read and easy to interact with. This may be of particular importance for old patients, and/or unwell patients with comorbidities where a large screen more clearly presents the queries and answers relative to a smaller screen.

In one example the touchscreen comprises a resolution is at least 300×150 pixels. In one example the touchscreen comprises a resolution of 400×250 pixels. More preferably the screen comprises a resolution of 480×272 pixels.

The touchscreen may have a resolution of at least 600×400 pixels

The touchscreen may be at least 3.5 inches in diagonal measurement.

The touchscreen may be at least 4 inches in diagonal measurement.

The touchscreen may be 4.2 inches in diagonal measurement.

The touchscreen may be up to 7 inches in diagonal measurement.

The touchscreen may be an OLED or TFT LCD screen.

In an alternative form, the display may comprise a colour touchscreen with a plurality of buttons or mechanical switches. The touchscreen in combination with the touchscreen allows a patient to input information using a combination of buttons or switches or dials and the touchscreen.

As shown in FIG. 3, the apparatus 100 includes a shroud 190 that supports the screen 212 and fits about the elbow 30. The shroud 190 is designed to not be removed during regular use. The shroud has features that allow it to be clipped onto a screen carrier, which in turn is fastened to the upper chassis 102 to become part of the housing 100. The screen carrier can connect to and support a display 212. In alternative configurations, the screen carrier may not be provided, and the shroud 190 may clip directly to part of the housing 100, such as an upper surface or upper chassis 102 of the housing.

The shroud 190 is configured so that the shroud cannot be detached from the screen carrier of the housing solely by pulling the removable elbow 171 in the second forwards direction relative to the housing 100. The removable elbow 171 is removable from the housing 100 when the shroud 100 is attached to the housing. The breathing assistance apparatus 10 may comprise the features of a breathing assistance apparatus, in particular a high flow apparatus as described in U.S. 62/925,971, the contents of this specification are incorporated herein in its entirety by way of reference.

The apparatus may comprise a valve to allow supplementary gases to be introduced to the blower.

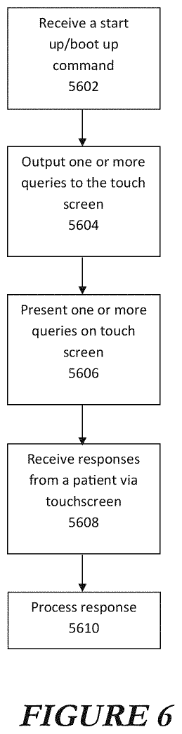

FIG. 5 is a schematic of a breathing assistance apparatus. In particular, a breathing assistance apparatus (the breathing assistance apparatus may be the breathing assistance apparatus 10 as described above) can be similar to other respiratory or breathing apparatuses described above. The respiratory apparatus 5700 includes a housing 5702 (e.g., housing 100) including a controller 5704 (e.g., controller 13), a switch 5706, a speaker 5708, a flow generator 5710 (e.g., flow generator 11), a touch-enabled screen 212, a network interface 5714, and a humidifier 5716 (e.g., humidifier 12).

As described above in context of FIGS. 1 to 3, the housing 5702 has a fluid inlet and a fluid outlet. Likewise, the flow generator 5710 is located within the housing 5702 downstream of and in fluid communication with the fluid inlet, whether mains or battery powered. Similarly, the humidifier 5716 is located within the housing 5702 downstream of and in fluid communication with the flow generator 5710 and upstream of and in fluid communication with the fluid outlet, whether mains or battery powered. Moreover, the humidifier 5716 including a heater (e.g., heating plate, heating element), whether mains or battery powered.

The controller is in electronic communication with the flow generator, the display, the network interface, and the heater plate. The controller comprising an electronic processor (e.g., logic controller, multicore processor) and a non-transitory memory (e.g., flash memory) in communication with the electronic processor. The controller controls activation/deactivation and operation of the flow generator, the heater plate, and the screen 212.

The switch 5706 (e.g., analog, digital) is located on the housing 5702, but can be located not on the housing 5702 (e.g., on power cord), or can be incorporated as a software implement switch on, (e.g., screen 212). The switch 5706 is a power switch electrically and/or mechanically coupled to the controller 5704 and that switches between an on-mode and an off-mode, which can be based on a manual input (e.g., user input, patient input). The respiratory apparatus 5700 can be activated based on the switch 5706 switching from the off-mode to the on-mode and deactivated based on the switch 5706 switching from the on-mode to the off-mode. The switch 5706 can be embodied in many physical, electronic, or virtual ways. For example, the switch 5706 can be embodied as a physical or virtual button, a knob, a dial, a rocker, a toggle, or a lever. The switch 5706 can be omitted.

The speaker 5708 is located on the housing 5702 and is configured to output a sound content (e.g., tones, speech, music). The speaker 5708 can be mono or stereo. The speaker 5708 can be mains or battery powered. In some embodiments the speaker 5708 is not present.

The display 5712 (for example screen 212) is located on the housing 5702 (e.g., upper side, lateral side) and is positionally fixed. However, the display 5712 can be positionally adjustable (e.g., hingedly). The screen 212 can be grayscale or color. The screen 212 can be a touchscreen (e.g., resistive, capacitive). The screen 212 is rectangular, but can be shaped differently (e.g., square, circular). The screen 212 is mains or battery powered.

In some embodiments the apparatus allows for insertion of a USB or memory storage device. This allows for data (e.g. responses to the queries) to be downloaded and then plugged into a computer, such as a PC or laptop. The PC or laptop can then be used to transmit responses to the patient and device platform e.g. a server for patient and device management.

In some embodiments the apparatus comprises a network interface. The network interface is located within the housing, but can be located externally on the housing. The network interface includes a wireless signal receiver, a wireless signal transmitter, or a wireless signal transceiver, each having a wireless signal radio antenna or a light signal modulator depending on signal modality (e.g., radio, light), although wired communication is possible (e.g., wired network card). The network interface is configured to communicate on a Wi-Fi, Li-Fi, Bluetooth, ZigBee, Z-Wave, cellular, or a satellite network, whether via a local, wide, personal or other area networks. For example, the network interface can communicate with a patient and device management platform e.g. a server which may or may not be a patient and device management platform (e.g., web, application, database, virtual server, cloud service) or a computing device (e.g., smartphone, tablet, wearable, medical monitor) that is local or remote to the network interface. Note that the network interface can include a first transceiver of a first modality (e.g., Wi-Fi) and a second transceiver of a second modality (e.g., Bluetooth). For example, the network interface can include a cellular modem (3G, 4G, 5G, 6G), a Wi-Fi card, and a Bluetooth chip. In some embodiments the network interface is not present.

The memory of the controller includes instructions executable by the electronic processor that when executed by the electronic processor cause the controller to perform various operations, as further described below.

For example, the instructions can cause the controller to (a) activate the heater plate upon activation of the breathing assistance apparatus, (b) request the screen 212 to display a user interface presenting a plurality of user health queries (e.g., relating to patient disease progression or patient health condition) and a plurality of user input elements via which user inputs are received, (c) refrain from activating or prevent activation of the flow generator until a predetermined plurality of the user inputs have been received, and (d) send the predetermined plurality of user inputs to a patient and device management platform (e.g. a server, webapplication, database, virtual server, cloud service) via the network interface. The controller can receive the predetermined plurality of user inputs before the heater plate of the humidifier reaches a predetermined temperature (e.g., about 37 degrees temperature of gas). The user health queries and the user input elements can be displayed upon booting of the controller.

For example, the instructions can cause the controller to (a) request the screen 212 to display a user interface presenting a plurality of requests for user health information and a plurality of user input elements via which the user health information is received by the controller as user inputs and (b) refrain from activating or prevent activation (e.g., hardware, software) of the flow generator or the heater plate, which can be upon booting of the controller, until a predetermined plurality of the user inputs from the user input elements are received. The requests for user health information and the user input elements can be displayed upon booting of the controller. The predetermined plurality of the user inputs can be all of the user inputs. The controller can refrain from activating or prevent activation of the flow generator and/or the heater until the predetermined plurality of the user inputs from the user input elements are received unless the requests for user health information and the user input elements are user bypassed (e.g., hardware, software). The predetermined plurality of the user inputs is all of the user inputs. The controller can refrain from activating or prevent activation (e.g., hardware, software) of the flow generator or the heater plate based on the controller employing or electrically communicating with a valve (e.g., solenoid valve) or a gate of the breathing assistance apparatus to be in an off position, a block flow position, or a restrict flow position. The controller can enable bypass/override for a predetermined time period (e.g., once per day for tracking purposes over 30 or 60 days).

For example, the instructions can cause the controller to refrain from activating or prevent activation (e.g., hardware, software) of the flow generator or the heater plate, which can be upon booting of the controller, until a predetermined plurality of user inputs responsive to a plurality of displayed requests for user health information are received via the display unless the displayed requests for user health information are user bypassed (e.g., hardware, software). The requests for user health information and the user input elements can be displayed upon booting of the controller. The predetermined plurality of the user inputs can be all of the user inputs. The controller can refrain from activating or prevent activation of the flow generator or the heater until the predetermined plurality of the user inputs from the user input elements are received unless the requests for user health information and the user input elements are user bypassed (e.g., hardware, software). The predetermined plurality of the user inputs is all of the user inputs. The controller can refrain from activating or prevent activation (e.g., hardware, software) of the flow generator or the heater based on the controller employing or electrically communicating with a valve (e.g., solenoid valve) or a gate of the breathing assistance apparatus to be in an off position, a block flow position, or a restrict flow position. The controller can enable bypass/override for a predetermined time period (e.g., once per day for tracking purposes over 30 or 60 days).

The controller may be configured to communicate two or more queries sequentially to the screen 212, the screen 212 presenting the two or more queries sequentially to the user. After receiving a response to each presented query at the screen 212, the screen 212 is configured to communicate each response to the controller.

The controller may also be configured to lock access to any other modes, functions until the controller receives a response to each query. Alternately, the controller may restrict access to an operative mode until the controller receives response to each query. Alternately, the controller may be configured to disable operation of the flow generator and heater plate until a response to each query is received. However the controller may allow a user bypass responding to the enquiry or may automatically bypass presenting the enquiry if the user i.e. patient has responded to the enquiry at least once in a day.

FIG. 6 is a flowchart for the controller operation. As shown in block 5602 of FIG. 6 the breathing assistance apparatus may receive a start up/boot up command which initiates the device. The start up/boot up command may be received by the apparatus via the user interface of the screen 212. In some embodiments, when power is provided to the apparatus the screen is configured to be activated, so that the user can interact with the screen. Additionally or alternatively, the start up/boot up command may be received by the apparatus via a switch (for example a button).

This start-up can include booting of the breathing assistance apparatus or activation of the breathing assistance apparatus. As indicated by block 5604, the start up/boot up activates the apparatus to initiate the enquiry to the user which leads to one or more queries being output to the screen 212 so that the one or more queries are presented on the touch screen for the patient (see block 5606).

In some embodiments, entering warm up mode and/or drying mode activates the apparatus to initiate the enquiry to the user.

In some embodiments, as described in more detail above the start-up may include presenting a therapy control screen, which the patient may use to initiate therapy.

The queries can have a first query and a second query, where the first query precedes the second query. Correspondingly, the potential answers include a first potential answer input and a second potential answer, where the first potential answer precedes the second potential answer and the first potential answer corresponds to the first query and the second potential answer corresponds to the second query. As such, the second query can be content-dependent on the first answer, whether the first potential answer and the second potential answer are from a same user session or not (e.g., the first potential answer is from a first user session and the second potential answer is from a second user session, where the first user session precedes the second user session). For example, this content dependency can be based on a for loop, a while loop, a counter, if-then logic tree, or other logical expressions, whether the second query is retrieved or generated from the memory of the breathing assistance apparatus 5700 (the breathing assistance apparatus 5700 may be the breathing assistance apparatus 10 as described above) or from the patient and device management platform e.g. a server in communication with the breathing assistance apparatus 5700.

In some embodiments, the answer to an earlier query (for example the first query) may determine the content of a subsequent query (for example the second query). For example the answer to earlier queries may update the particular queries in the health enquiry, and/or the update order of the queries in the queries in the health enquiry.

In some embodiments, after a query is answered (for example a first query) a health parameter may be updated, the determination of the content of a subsequent query (for example the second query) may be based on the health parameter (for example the updated health parameter.)

The health provider (for example a doctor) may be able to generate a health enquiry for a patient by selecting one or more predetermined queries from a database of queries.

The database of queries may be presented to the health provider as a list (for example a tick box interface) and the health provider can select a number of queries from the database of queries to form the enquiry.

The database of queries may be located on the patient and device management platform and/or the apparatus.

In some embodiments the database of queries may be customised based on a worsening of one or more symptoms. For example if a patient is indicating a worsening in symptoms related to sputum colour the database of queries may be customised to include queries related to upper airway health.

If the health provider (for example a doctor) is selecting the one or more predetermined queries from a database of queries remotely (i.e. not at the apparatus) once the healthcare provider selects the queries, the selected queries may be transmitted to the apparatus, and stored on the device.

In some embodiments, the apparatus may periodically communicate with the patient and device management platform to obtain any updates to the enquiry and/or any queries. Additionally, or alternatively, the patient and device management platform may communicate with the apparatus to notify the apparatus that updates to the enquiry and/or any queries are available, and transmitted the updated enquiry and/or queries.

In some embodiments the queries (as part of the health enquiry) may be based on a patient condition (for example COPD, bronchiectasis etc). Queries may be added to, or removed from the health enquiry based on the patient condition. For example if the patient has bronchiectasis then the health enquiry may include an enquiry relating to the colour of sputum and whether the patient is taking antibiotics.

The health provider (for example a doctor) may select queries from a list relating to a patient condition to be added to the health enquiry. Alternatively or additionally, the health provider (for example a doctor) may select a patient condition and the health enquiry may be automatically updated based on the selection. In some embodiments, the database of queries may be customised based on a patient condition (for example chronic obstructive pulmonary disease (COPD) or respiratory distress syndrome or dyspnea, or bronchiectasis etc.) For example if the patient has COPD the database of queries may be customised to include queries relating to COPD.

In some embodiments, the queries of the health enquiry are ordered such that queries related COPD condition are displayed first, followed by queries related to a bronchiectasis condition.

In some embodiments, general health queries are presented before queries related to a COPD condition and queries related to a bronchiectasis condition.

For example queries relating to COPD may include: daily sputum production, what colour is your sputum, and/or how is your cough.

For example queries relating to bronchiectasis may include: Are you taking steroids, and/or are you using your inhaler.

In some embodiments the specific condition (for example COPD or bronchiectasis) of the patient may be determined based on the answers to queries relating to the specific condition. For example if the answers to the queries relating to bronchiectasis are below a baseline and/or worsening this may be indicative of the patient having bronchiectasis.

The health provider (for example a doctor) may be able to add custom queries to the health enquiry. The custom enquires may include custom questions and associated answers relating to information the doctor may wish to keep track of.

The health provider (for example a doctor) may be able to add custom queries to the database of queries.

The health provider may generate the health enquiry directly on the apparatus or from an ancillary device (such as a mobile device connected to the patient and device management platform).

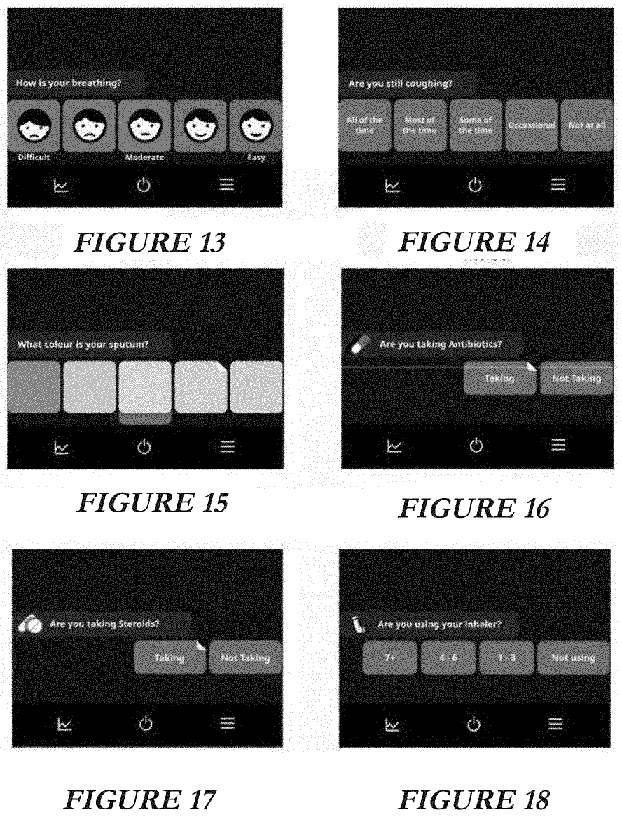

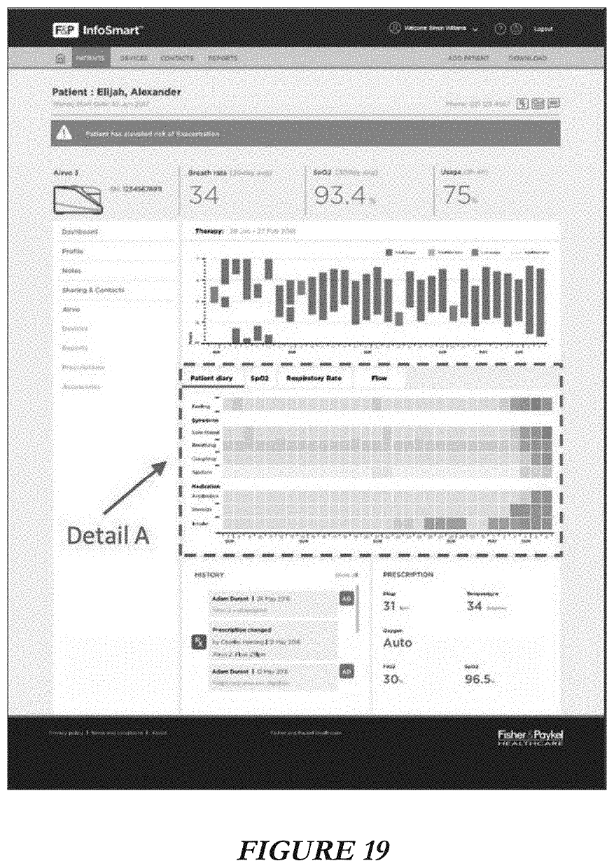

The patient then enters their response via the touch screen to each query (see block 5608). The patient's responses are then processed 5910. This processing may include plotting the set of the responses, as shown in FIGS. 19 and 20, or sending the data set of responses to a patient and device management platform, external storage, mobile device, or insurance, equipment or healthcare provider. Analysis or processing of the data set can be performed at any stage, for example, by the patient and device management platform. In some embodiments, after the patient answers the last query of the enquiry, the breathing assistance apparatus displays a therapy control screen, which the patient may use to initiate therapy.

In some embodiments, the patient and device management platform can store a patient profile that comprises one or more of: patient details, baseline data of the health parameters of a patient, serial number of the patient's Airvo device and/or prescription settings.

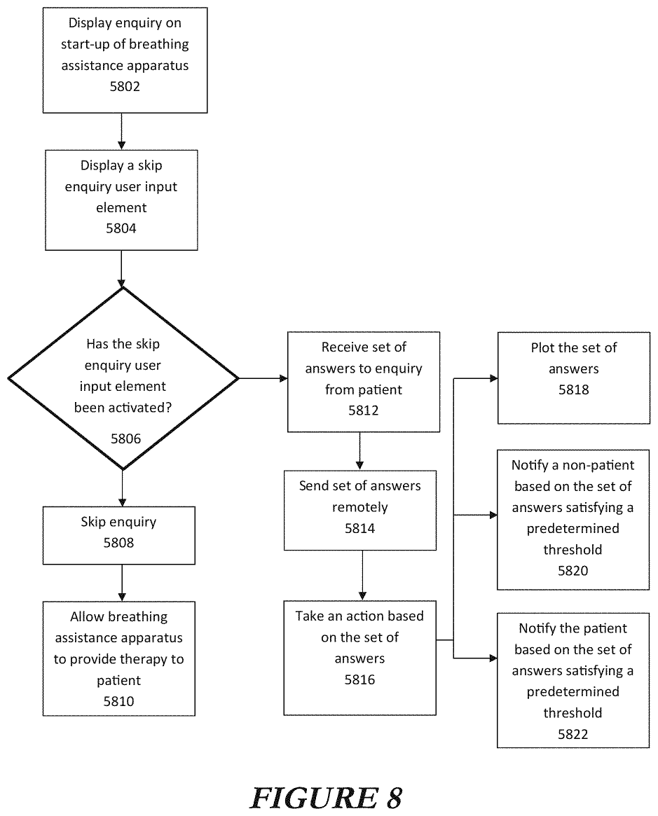

FIG. 8 is a flowchart of a process for conditionally presenting a personal health enquiry to a patient of a breathing assistance apparatus. In particular, a process 5800 is performed via the breathing assistance apparatus 5700 and the patient and device management platform, as described above. The breathing assistance apparatus 5700 may be the breathing assistance apparatus 10 as described above.

The term block in the specification may refer to one or more steps undertaken by the apparatus 5700 (for example by the controller).

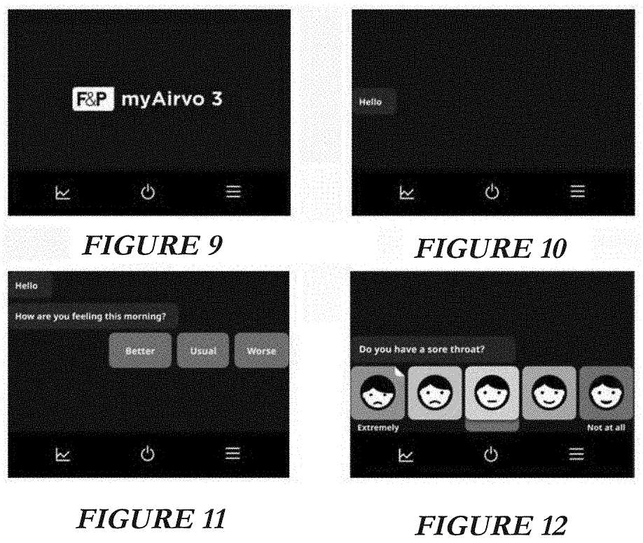

In block 5802, the breathing assistance apparatus 5700 (the breathing assistance apparatus 5700 may be the breathing assistance apparatus 10 as described above)(e.g., controller 5704) displays a plurality of health queries as a personal health enquiry, as shown in FIGS. 9 to 18, on the display 5712 (same as display 212) via the controller 5704 (same as controller 13) to a patient (or if the patient is incapable a caretaker or doctor) upon start-up of the breathing assistance apparatus 5700. This start-up can include booting of the breathing assistance apparatus 5700 (e.g., controller 5704) or activation of the breathing assistance apparatus 5700 (e.g., flow generator 5710, humidifier 5716) prior to use if the breathing assistance apparatus 5700 (e.g., controller 5704) is already booted and running. In some embodiments start-up may include a warm up process (as described above.)

In situations where the network interface 5714 is absent or the networking interface 5714 is unable to establish a network connection, the controller 5704 can still present the personal health enquiry and the answers can be later downloaded onto a removable memory (e.g., flash card, flash drive) or retrieved from the memory of the breathing assistance apparatus 5700 itself (e.g., maintenance). The humidifier 5716 may be the same as humidifier 12 as described earlier. The flow generator 5710 may be the same as flow generator 11 described earlier.

In block 5804, the breathing assistance apparatus 5700 (e.g., controller 5704) displays a skip enquiry user input element (e.g., graphic, text, icon) on the display 5712 via the controller 5704. Alternatively, the controller 5704 can determine whether the activation is a second or greater activation within a predetermined interval, e.g., a day, and then effect a bypass of presentation of the mandatory enquiry. Further, the controller 5704 can be programmed to request the display 5712 (e.g. a touchscreen like screen 212) to present the queries and the potential answers such that at least one of the queries or at least one of the potential answers is different between at least two instances of the flow generator 5710 being activated over a predetermined time period (e.g., at least two days).

In block 5806, the breathing assistance apparatus 5700 (e.g., controller 5704) determines (e.g., controller 5704) whether the skip enquiry user input element has been activated (e.g., via touch selection). If yes, then block 5808 is performed. If no, then block 5812 is performed. Note that this skipping functionality can be employed on a per enquiry basis (e.g., skip entire enquiry if the patient is feeling too unwell to complete it) or on a per query basis (e.g., skip specific or any queries).

Skipping information may be tracked, which itself may be a data point later identified by the patient and device management platform e.g. server.

Further, in certain situations, skipping is not enabled or is prevented. For example, if the enquiry is completed at the time the breathing assistance apparatus 5700 is first started or booted during a calendar day, then the controller 5704 will allow skipping of the query or the enquiry if the breathing assistance apparatus 5700 is switched off and started a second time in that calendar day. In some embodiments, the enquiry may need to be completed once a calendar day as controlled by the controller 5704.

In certain situations, a patient and device management platform e.g. server may send a signal to allow the controller 5704 to skip if the server has received the answers to the queries at least once a day. In certain situations, the queries may be required to be completed at a predefined time intervals (e.g. every 2 days or every 3 days) or could be physician set or may be defined by clinical practice.

In block 5808, the breathing assistance apparatus 5700 (e.g., controller 5704) skips the personal health enquiry via the controller 5704 and presents a menu via the display 5712 for control (e.g., via touch selection) of the breathing assistance apparatus 5700 (e.g., flow generator 5710, humidifier 5716). For example, this allows skipping the enquiry before activating the flow generator 5710. However, the controller 5704 can be programmed to prevent or preclude skipping the enquiry before activating the flow generator 5710.

In block 5810, the breathing assistance apparatus 5700 (e.g., controller 5704) allows the flow generator 5710 or the humidifier 5716 to be used for provision of a breathing assistance apparatus to the patient. For example, the patient can operate the menu via the display 5712 to control (e.g., via touch selection) of the breathing assistance apparatus 5700 (e.g., flow generator 5710, humidifier 5716), whether to start the breathing assistance apparatus, modify the breathing assistance apparatus, or end the breathing assistance apparatus.

In block 5812, the breathing assistance apparatus 5700 (e.g., controller 5704) receives a set of responses or answers to the personal health enquiry from the patient. Such receipt can be performed via the patient touch selecting various user input elements (e.g., graphics, text, icons) displayed on the display 5712 via the controller 5704.

In block 5814, the breathing assistance apparatus 5700 (e.g., controller 5704) sends (e.g., wired, wireless, waveguide, encrypted, decrypted, unencrypted) the set of answers via the network interface 5714 to the patient and device management platform. The patient and device management platform may be any suitable platform for example a server web application, database, cloud service, a virtual server. The patient and device management platform is remote from the breathing assistance apparatus 5700 such that the patient and device management platform can receive the set of answers and process the set of answers, as described below. The controller 5704 can request the network interface 5714 to send the set of answers (e.g., predetermined plurality of user touch inputs) to the patient and device management platform one-by-one after each answer of the set of answers is received via the controller 5704 (e.g., answer-send followed by another answer-send). The controller 5704 can request the network interface 5714 to send the set of answers (e.g., predetermined plurality of user touch inputs) to the patient and device management platform after all of the answers (e.g., user touch inputs) are received via the controller 5714 (e.g., single send operation, single packet). The controller 5704 can request the network interface 5714 to send the set of answers (e.g., predetermined plurality of user touch inputs) to the server on a group-basis after a group of the answers is received via the controller (e.g., send after every two, three, four, five, six, seven etc. answers). Note that the controller 5704 can request the network interface 5714 to send the set of answers with a patient or machine identifier in order to enable effective patient and device management platform based identification of the set of answers and subsequent processing.

In some embodiments, the set of answers is stored in memory of the apparatus. Optionally the set of answers may be provided to the patient and device management platform.

In some embodiments the apparatus may be configured to store answers, and transmit answers to the patient and device management platform once every predetermined time period (for example once a day). This may lead to lower data transmission costs relative to transmitting at a higher frequency as the data transfer required to generate a connection between the apparatus and the patient and device management platform needs to be undertaken less frequently.

In some embodiments the apparatus may be configured to store answers, and transmit answers to the patient and device management platform when a drying process is activated (either automatically or by the user).

In block 5816, the patient and device management platform takes an action based on the set of answers being processed. For example, such action can include identifying the patient or machine identifier (e.g., alphanumeric, barcode) associated with the patient answers, matching the patient or machine identifier associated with the patient answers with a patient or machine identifier (e.g., alphanumeric, barcode, or serial number) stored via or accessible to the patient and device platform, locating a patient or machine profile(e.g., data structure, database record) based on the patient or machine identifier, writing the patient answers to the patient or machine profile, reading the patient or machine profile including the patient answers, and performing an analytic (e.g., plotting patient answers against time, predicting non-change or positive or negative change to patient health condition based on patient answers) on the patient or machine profile, as further described below. For example, the patient and device platform may merge i.e. fuse enquiry data (e.g. data provided from the answers to the enquiry) and sensor data (e.g., sourced from the breathing assistance apparatus 5700), and such fusion can be actionable. For example, the patient and device management platform e.g. a server can be programmed to detect change to answers to queries over time and make that a data point, which can be actionable.

In some embodiments, the sensor data may be one or more patient parameters (for: respiratory rate, oxygen saturation of the patient as described in more detail elsewhere in the specification).

As used herein, a patient's “health condition” is a collective determination of the patient's health based on the health parameters of the patient.

In block 5818, the action can include the server plotting the set of answers, as shown in FIGS. 19 and 20. The plot can provide a graphical representation of the patient's health parameters and change in the patient's health parameters. The plot can also indicate a patient health condition.

The plot may be part of a report presented to a user (for example a patient, caretaker, or healthcare provider).

In some embodiments the set of answers, and optionally a plurality of answers or sets of answers to a plurality of queries of a personal health enquiry (for example including historic health enquiries which may be stored in memory of the apparatus and/or the patient and device management platform) may be plotted and/or form part of the report.

In some embodiments, the apparatus is configured to generate and/or display the report on the screen.

In some embodiments, the patient and device management platform is configured to generate the report, and the apparatus is configured to display the report on the screen of the apparatus.

In some embodiments, the patient and device management platform is configured to generate the report, and the apparatus is configured to provide the report to a healthcare provider.

In block 5820, the action can include the patient and device management platform e.g. server notifying non-patient (e.g., family member, doctor, caretaker) via a computing device (e.g. smartphone, tablet, wearable, workstation) in signal communication (e.g., wired, wireless, waveguide) with the patient and device management platform e.g. a server. Such notice can include text, graphics, audio, video, or other forms of content. For example, such notice can be manifested via a mobile app, a dedicated software application, a browser-navigable portal, or other forms of software. The notice can be prompted or generated based on the set of answers satisfying or non-satisfying a predetermined threshold, as further described below. For example, the threshold can be a baseline custom set by a doctor of the patient and the threshold can be satisfied based on a predetermined deviation from the baseline.

In some embodiments, the baseline may be set by the doctor (or other healthcare provider) based on an initial set of tests of the patient.

In block 5822, the action can include the patient and device platform e.g. a server notifying the patient (or caretaker in care proximity of patient) via a computing device (e.g. smartphone, tablet, wearable, workstation) in signal communication (e.g., wired, wireless, waveguide) with the patient and device platform e.g. server or the breathing assistance apparatus 5700 in signal communication with the server via the network interface 5714. Such notice can be generated based on the set of answers and can include text, graphics, audio, video, or other forms of content. For example, such notice can be manifested via a mobile app, a dedicated software application, a browser-navigable portal, or other forms of software. For example, such notice can be output via the speaker 5708 or the display 5712. The notice can be prompted or generated based on the set of answers satisfying or non-satisfying a predetermined threshold, as further described below. For example, the threshold can be a baseline custom set by a doctor of the patient and the threshold can be satisfied based on a predetermined deviation from the baseline. In some embodiments, when notice may be provided on the apparatus, or to a healthcare provider.

After the enquiry has been completed, the controller 5704 can control the display 5712 to present a screen (e.g., page) after the controller 5704 receives the set of answers, whether before, during, or after sending to the patient and device management platform e.g. server. The screens presents a menu to control or activate the flow generator 5710 or the humidifier 5716 or to input an operational parameter of the flow generator 5710 or the humidifier 5716. Note that although the process 5800 is performed via the breathing assistance apparatus 5700 and the patient and device management platform , in certain situations, the process 5700 can be performed locally. For example, instead of sending the set of answers to the patient and device management platform, the breathing assistance apparatus 5700 (i.e. breathing assistance apparatus 10) can be programmed to perform similar processing locally and then take actions, as described herein. For example, the breathing assistance apparatus 5700 can perform blocks 5820 or 5822.

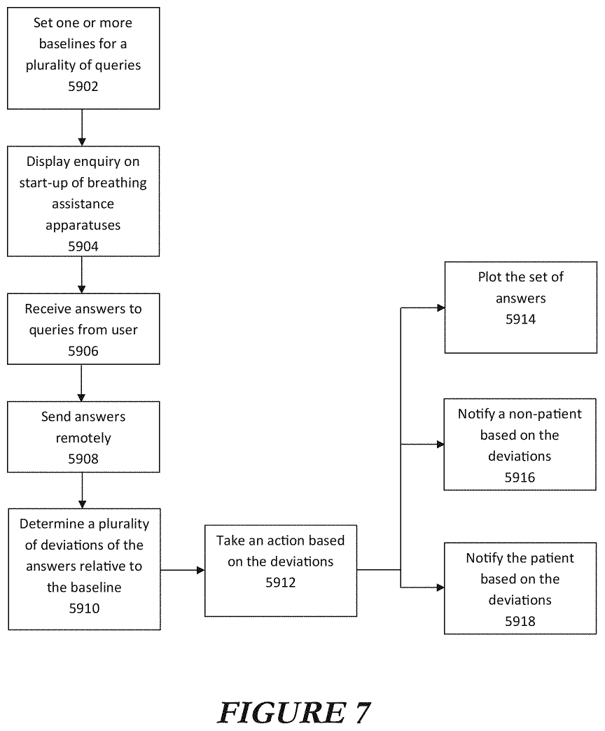

FIG. 7 is a flowchart of a process for determining deviations to answers of a personal health enquiry based on preset baselines. In particular, a process 5900 is performed via the breathing assistance apparatus 5700 and the server, as described above.

In block 5902, a user (e.g., doctor, nurse) operates a computing device (e.g., smartphone, tablet, wearable, workstation) in signal communication (e.g., wired, wireless, waveguide) with a patient and device management platform (e.g. a server web, application, database, cloud service). As such, the user accesses (e.g., browser session, dedicated software application session) a patient or machine profile via the server and sets a plurality of baselines for a plurality of queries of a personal health enquiry associated with the patient or machine identifier. For example, the patient and device platform e.g. server can present a browser-based user interface to the computing device operated by the user, where the user interface is programmed to receive a plurality of user inputs (e.g., alphanumerical, binary, or Boolean values entered or selected via textboxes, checkboxes, dropdown menus, radio buttons, sliders, dials) corresponding to the baselines as the baselines one-to-one correspond to the queries such that answers to the queries can be subsequently measured against the baselines, as set by the user.

In block 5904, the breathing assistance apparatus 5700 (e.g., controller 5704) displays the personal health enquiry, as shown in FIGS. 9 to 18, on the display 5712 via the controller 5704 to a patient (or caretaker or doctor) upon start-up of the breathing assistance apparatus 5700. This start-up can include booting of the breathing assistance apparatus 5700 (e.g., controller 5704) or activation of the breathing assistance apparatus 5700 (e.g., flow generator 5710, humidifier 5716) prior to use if the breathing assistance apparatus 5700 (e.g., controller 5704) is already booted and running. The enquiry comprises one or more queries that are presented onto the screen 212 i.e. display 5712.

In block 5906, the breathing assistance apparatus 5700 (e.g., controller 5704) receives a plurality of answers to the personal health enquiry from the patient. Such receipt can be performed via the patient touch selecting various user input elements (e.g., graphics, text, icons) displayed on the display 5712 via the controller 5704. The patient may also respond to the queries by performing a gesture or touching a portion of the display or pressing a portion of the display.

In block 5908, the breathing assistance apparatus 5700 (e.g., controller 5704) sends (e.g., wired, wireless, waveguide, encrypted, decrypted, unencrypted) the answers via the network interface 5714 to the patient and device management platform (e.g. a server) remote from the breathing assistance apparatus 5700 such that the patient and device management platform can receive the answers and process the answers, as described below. The controller 5704 can request the network interface 5714 to send the answers (e.g., predetermined plurality of user touch inputs) to the server one-by-one after each of the answers is received via the controller 5704 (e.g., answer-send followed by another answer-send). The controller 5704 can request the network interface 5714 to send the answers (e.g., predetermined plurality of user touch inputs) to the patient and device management platform e.g. server after all of the answers (e.g., user touch inputs) are received via the controller 5714 (e.g., single send operation, single packet). The controller 5704 can request the network interface 5714 to send the answers (e.g., predetermined plurality of user touch inputs) to the patient and device management platform e.g. server on a group-basis after a group of the answers is received via the controller (e.g., send after every two, three, four, five, six, seven etc. answers). Note that the controller 5704 can request the network interface 5714 to send the answers with a patient or machine identifier in order to enable effective server-based identification of the answers and subsequent processing.

In block 5910, the patient and device management platform e.g. server receives the answers from the network interface 5714, identifies the patient or machine identifier (e.g., alphanumeric, barcode) associated with the patient answers, matches the patient or machine identifier associated with the patient answers with a patient or machine identifier (e.g., alphanumeric, barcode) stored via or accessible to the server, locates a patient or machine profile(e.g., data structure, database record) based on the patient or machine identifier, writes the patient answers to the patient or machine profile, retrieves the baselines that have been previously set, reads the patient or machine profile including the patient answers, performs a comparison between the baselines and the answers (e.g., baselines to answers or answers to baselines), and determines a plurality of deviations (e.g., binary value, Boolean value, alphanumeric value) of the answers relative to the baselines, if such deviations exist. Note that some of the deviations can include a degree of deviation (e.g., up by 5 points relative to baseline or down 10% relative to baseline).

Alternatively the processes of determining if a plurality of deviations of the responses relative to a baseline may be implemented and executed by the controller of the breathing assistance apparatus instead of the patient and device management platform.

In some embodiments, in block 5910, the patient and device management platform and/or the apparatus may determine deterioration of patient health.

The patient may be considered to be unstable if their health parameters are not stable.

Deterioration of patient health may be indicative of the patient being at risk of an onset of an exacerbation, or being at risk of an exacerbation.

The exacerbation may be a COPD exacerbation.

The deterioration of patient health of may be indicative of one other health issues for example a cold, hay fever, an allergic reaction etc.

The indication of a deterioration of patient health may allow for a health provider to undertaken further

The patient and device management platform and/or the apparatus may determine instability in patient health based on the answers to user queries, and one or more historic answers to the user queries to determine a worsening

The patient may be determined to be deteriorating if there is a worsening of at least one health parameter (optionally relative to a baseline).

The patient may be determined to be deteriorating if there is a worsening of two or more health parameters for at least two days (optionally relative to a baseline).

Additionally or alternatively, the patient may be determined to be deteriorating if there is a worsening in one or more patient parameter and/or one or more patient parameter drops below a threshold and/or one or more patient parameter changes by more than a threshold (for example blood oxygen concentration or respiratory rate).

The patient may be determined to be deteriorating if there is a worsening in one or more patient parameter (for example blood oxygen concentration or respiratory rate) from a baseline.

For example the patient may be determined to be deteriorating if SpO2 gets worse or drops below a threshold of for example 85%.

For example the patient may be determined to be deteriorating if respiratory rate increases by a threshold of for example 25%

In some embodiments, the patient may be determined to be deteriorating if for at least two days there is both:

a worsening of one or more health parameters (optionally from a baseline).

a worsening in one or more patient parameter (optionally from a baseline) and/or one or more patient parameter drops below a threshold and/or one or more patient parameter changes by more than a threshold.

For example the SpO2 and respiratory rate examples above may additionally require a worsening of at least one health parameter for the patient to be determined to be deteriorating.

In block 5912, the patient and device platform e.g. server takes an action based on the deviations (or absence thereof) being processed. For example, the patient and device platform can perform an analytic (e.g., plotting patient answers against time, predicting non-change or positive or negative change to patient health condition based on patient answers) on the patient or machine profile, as further described below.

In block 5914, the action can include the server plotting the set of answers, as shown in FIGS. 19 and 20, such that the deviations are visually identifiable or visually distinct.

In block 5916, the action can include the patient and device platform e.g. server notifying non-patient (e.g., family member, doctor, caretaker) via a computing device (e.g. smartphone, tablet, wearable, workstation) in signal communication (e.g., wired, wireless, waveguide) with the server. Such notice can be generated based on the deviations (or absence thereof) and can include text, graphics, audio, video, or other forms of content. For example, such notice can be manifested via a mobile app, a dedicated software application, a browser-navigable portal, or other forms of software.

In block 5918, the action can include the patient and device platform e.g. server notifying the patient (or caretaker in care proximity of patient) via a computing device (e.g. smartphone, tablet, wearable, workstation) in signal communication (e.g., wired, wireless, waveguide) with the server or the breathing assistance apparatus 5700 in signal communication with the server via the network interface 5714. Such notice can be generated based on the deviations (or absence thereof) and can include text, graphics, audio, video, or other forms of content.

In some embodiments, the action can include notifying the patient and/or notifying non-patient (e.g., family member, doctor, caretaker) based on a determination that the patient is deteriorating.

In situations where patient or caretaker notification is desired, then the network interface 5714 can receive a message (patient or caretaker notice) from the server, where the message is based on a predetermined plurality of user inputs (patient enquiry answers) previously sent to the server via the network interface 5714. For example, such notice can be manifested via a mobile app, a dedicated software application, a browser-navigable portal, or other forms of software. The message can include a video content for output via the display 5712. When the housing 100 houses the speaker 5708, then the message can include an audio content for output via the speaker 5708.

In situations where the breathing assistance apparatus 5700 has a plurality of network interfaces 5714 (e.g., Wi-Fi, Bluetooth), the breathing assistance apparatus 5700 can send a second message (e.g., patient or caretaker notice) to a computing device (e.g., smartphone, tablet, medical accessory) responsive to the first message (patient or caretaker notice from server responsive to patient enquiry answers). The second message can be sent by the second network interface (e.g., Bluetooth) and still be associated with the predetermined plurality of user inputs sent to the server via a first network interface (e.g., Wi-Fi). The second network interface (e.g., Bluetooth) can be local to the computing device, where the computing device is other than the server.

The message from the patient and device management platform can be informative of the patient health parameter worsening over a predetermined time period (e.g., at least two, three, four, five, six, seven days) as determined based on at least one of the predetermined plurality of user inputs. For example, the message can be informative of the patient health parameter worsening relative to a baseline, which may or may not be factory-set. For example, as explained herein, the doctor may operating a computing device (e.g., phone, tablet, workstation) to set the baseline, where the computing device is in communication with the server, yet remote from the patient and device management platform e.g. server.

The message from the patient and device management platform may be a notification that the patient is deteriorating.

The steps of FIG. 7 have been described with reference to interactions between the breathing assistance apparatus and the patient and device management platform. However, in an alternative implementation steps described in blocks 5902, 5904, 5906, 5910, 5912 may be executed only by the breathing assistance apparatus 5700 (i.e. breathing assistance apparatus 10). For example the processing in block 5910 may be performed by the controller of the breathing assistance apparatus and the options in block 5912 may be performed by the controller of the breathing assistance apparatus. For example block 5918 may comprise presenting notifications to a patient upon the display of the breathing assistance apparatus.

In a further alternative implementation the responses to the queries can be received at the controller of the device 5906. The queries may be transmitted to a user device e.g. a tablet or a mobile phone or the queries may be downloaded onto a USB and then transferred to a laptop of PC for further processing. The block 5910 may be executed by a user device or by the laptop of PC. Further the functions described at block 5914 and 5918 may be executed by the user device or laptop or PC. The user device or laptop of PC may be in signal communication with a non patient device e.g. a physician server or physician computing device. The notifications at block 5916 may be provided by the user device or laptop or PC to the non patient device to notify a non patient about the patient health condition.

FIGS. 9 to 18 show a user interface presenting a personal health enquiry on a breathing assistance apparatus. The user interface includes a plurality of screens (e.g., pages) on which a plurality of queries and a plurality of potential answers are distributed. However, the user interface includes a single screen presenting the queries and the potential answers. Although FIGS. 9 to 18 show the user interface with a plurality of screens (e.g., pages) that are presented in an order, as shown in FIGS. 9 to 18, this order is illustrative and can vary. For example, the screen shown in FIG. 15 (or any other FIGS. 11 to 18) can be presented before the screen of FIG. 16 (or any other FIGS. 11 to 18) or after the screen of FIG. 18 (or any other FIGS. 11 to 18). The display 5712 displays the user interface with the screens of FIGS. 9 to 18, which are included in the personal health enquiry, as described herein. Further, FIGS. 9 to 18 can be embodied as a single screen that is vertically or horizontally touch scrollable (e.g., webpage manner). Also, the breathing assistance apparatus 5700 can be programmed (e.g., controller 5704) to repeatedly request the display 5712 to display the queries and the potential answers over a period of at least two consecutive days. Alternatively each query may be presented as a separate scrollable page.

FIG. 9 shows a booting screen that has a graph user touch element, a power user touch element, and a menu user touch element. For example, the booting screen can be a first screen that the patient sees when the patient turns on the breathing assistance apparatus, as described herein. The graph user touch element can enable a presentation of a data graph for various device parameters or patient health parameters when selected, as described herein, whether this presentation occurs on same page or on different page of the user interface. The power user touch element can be used to turn on or turn off or restart the breathing assistance apparatus when selected, as described herein. For example, responsive to selection of the power user touch element when the breathing assistance apparatus is already powered on, the power user touch element enable a display of a turn off graphic or a restart graphic on the display 5712. The menu user touch element can enable a presentation of a menu for various device usage options when selected, as described herein, whether this presentation occurs on same page or on different page of the user interface. The booting screen also shows a set of manufacturer and device identification information (e.g., text, graphics) above the graph user touch element, the power user touch element, and the menu user touch element. Note that the graph user touch element, the power user touch element, and the menu user touch element visually persist throughout the pages of the user interface shown in FIGS. 14 to 18, but can disappear for some pages of the user interface, whether all as a group or only some can disappear, but others remain.

FIG. 10 shows an introductory screen after the booting screen. The introductory screen displays a hello message (or some other introductory or welcoming message) above the graph user touch element, the power user touch element, and the menu user touch element, which visually persist from the booting screen (e.g., new screen with same graph user touch element, same power user touch element, and same menu user touch element or updating content above graph user touch element, power user touch element, and menu user touch element which remain displayed as is). The hello message indicates that the enquiry has started. Note that the hello message is started at a midway of a left vertical plane of the display 5712 to ease visibility, but this positioning can vary (e.g., non-midway, right vertical plane, spanning between left vertical plane and right vertical plane, top horizontal plane, display center).

FIG. 11 shows a general feeling screen that presents a query (e.g., request for user health information, health query) inquiring about a general feeling of the patient at a specific time of day. The query is alphanumeric, but can include pictorial content, whether additionally or alternatively. The query includes a concluding query mark, although this can be omitted (e.g., select one of following choices). The specific day of day is dynamic and changes based on when this query is displayed. This change can occur based on time/date tracked via the controller 5704 (e.g., internal clock). For example, a first set time period can correspond to morning (e.g., 4 AM to 11:59 AM of local time), a second set time period can correspond to afternoon (e.g., 12 PM to 5:59 PM of local time), and a third set time period can correspond to night (e.g., 6 PM to 3:59 AM of local time). This time of day dynamic change functionality can be omitted (e.g., how are you feeling now). Regardless, this enquiry is related to a general feeling parameter, as described herein, and an answer of ‘worse’ would suggest a worsening of the patient's health condition, whether this enquiry is related to a baseline or not.

The general feeling screen presents a group of user input elements (e.g., rectangular graphics containing embedded or overlaid text) associated with the query and representing a group of potential answers to the query, one of which can be selected exclusive to others (although this can vary in certain situations where multiple answers can be non-exclusively input). As shown, a first input element that denotes a current position or a non-change state of the health parameter (e.g., usual), a second input element that denotes an improvement in the health parameter (e.g., better), and a third user input element that denotes a deterioration of the health parameter (e.g., worse). The group of user input elements is between 2 and 9, although lower or higher numbers of user input elements are possible. The group of user input elements is a group of text strings and a group of graphics, whether defining a single group of content (e.g., text integrally embedded within graphics) or a plurality of groups of content where the group of text strings extends at least one of in the group of graphics or on the group of graphics (e.g., overlay). Regardless, although these user input elements are visually identical to each other but for potential answer content, at least two user input elements in the group of user input elements can be visually distinct from each other. For example, such visual distinction can be present based on graphic background or foreground color, graphic background or foreground shade of color, graphic shape, graphic size, font type, font size, font color, font shade of color, font arrangement, or other graphic or font characteristics.

The message of FIG. 10 and the query and the user input elements (potential answers) of FIG. 11 are presented in an electronic conversation like manner (e.g., chat style, email style, text message style) such that the query is positioned between the hello message and the potential answers. However, the electronic conversation like manner can be omitted or be presented in a different style (e.g., up/down or left/right swipe carousel). Although the query is presented to be visually distinct from the user input elements, this presentation can vary and such visual distinction can vary or be absent. The user interface can present the queries and the potential answers based on user scrolling via the touchscreen and the submitting the answers when the enquiry is completed.

FIG. 12 shows a sore throat screen that presents a query, similar to FIG. 15. However, unlike the query and the potential answers of FIG. 15, the query and the potential answers of FIG. 12 relate to a throat soreness parameter of the patient. The query of FIG. 12 comprises multiple possible answers each with an associated icon. The icons are colour coded relative to the patient condition associated with the answer, and have faces with expressions related to the patient condition associated with the answer (as described in more detail below). If the patient's doctor sets a baseline of, for example, the middle (light green) answer, then a patient's selection of anything to the left of that answer (the yellow or the orange answer) would suggest a worsening of the patient's health condition, whether this enquiry is related to a baseline or not. Note that the baseline potential answer is visually distinct (e.g., green) relative to other potential answers, although this can be absent. For example, such visual distinction can occur via overlaying the relevant user input elements over a graphic (e.g., green). Note that the user input elements include an odd number of user input elements, where the baseline is associated with a median user input element from the odd number of user input elements, although non-median user input element or even number of elements can be possible as well in any combination.

At least one user input elements (e.g., leftmost or rightmost) of the group of graphics corresponds to an alphanumeric text content other than the query (e.g., extremely and not at all) and that alphanumeric content is positioned external (e.g., graphic tooltip, graphic label) to the at least one user input element (e.g., vertical or horizontal orientation). Further, note that the user input elements are a first group of graphics (e.g., squares that are visually distinct from each other) that contain a second group of graphics therein (e.g., faces having different expressions), whether the second group of graphics is embedded or overlaid over the first group of graphics. Also, note that the first group of graphics are visually distinct from each other based on graphic background color although this can vary based on various characteristics (e.g. graphic shading, graphic hatching, graphic size, graphic shape) or avoid being visually distinct. Moreover, the second group of graphics are visually distinct from each other based on facial expression although this can vary based on various characteristics (e.g. face size, face shape, face color, face shade of color) or avoid being visually distinct from each other.

The faces as illustrated in FIG. 12 may have a range of expressions corresponding to different indications of comfort. For example the expressions of the faces may range from a sad or unhappy face (corresponding to a negative response to the query) to a smiling or happy face (corresponding to a positive response to the query). The expressions of the faces may have a neutral face (corresponding to a neutral response to the query).

FIG. 13 shows a breathing screen that presents a query, similar to FIGS. 11 and 12. However, unlike the queries and the potential answers of FIGS. 11 and 12, the query and the potential answers of FIG. 13 relate to a breathing parameter of the patient. If the patient's doctor sets a baseline of, for example, moderate (the middle answer), then anything to the left of that answer would suggest a worsening of the patient's health condition, whether this enquiry is related to a baseline or not.

FIG. 14 shows a coughing screen that presents a query similar to FIGS. 11 to 13. However, unlike the queries and the potential answers of FIGS. 11 to 13, the query and the potential answers of FIG. 14 relate to a coughing parameter of the patient. If the patient's doctor sets a baseline of, for example, most of the time, then anything to the left of that answer would suggest a worsening of the patient's health condition, whether this enquiry is related to a baseline or not.

FIG. 15 shows a sputum color screen that presents a query similar to FIGS. 11 to 14. However, unlike the queries and the potential answers of FIGS. 11 to 14, the query and the potential answers of FIG. 15 relate to a query relating to a sputum color parameter of the patient. If the patient's doctor sets a baseline of, for example, the color second from the right, then anything to the left of that answer would suggest a worsening of the patient's health condition, whether this enquiry is related to a baseline or not.

FIG. 16 shows an antibiotic use screen that presents a query similar to FIGS. 11 to 15. However, unlike the queries and the potential answers of FIGS. 11 to 15, the query and the potential answers of FIG. 16 relate to a query relating to an antibiotic use parameter of the patient. If the patient has not been taking antibiotics, then a selection of the taking answer would suggest a worsening of the patient's health condition, whether this enquiry is related to a baseline or not. Further, note that the potential answers are binary, although this can vary as needed. For example, FIG. 16 shows a pair of user input elements corresponding to the query relating to the antibiotic use parameter of the patient. The inputs from these user input elements are associated with a pair of data points that are mutually exclusive to each other (e.g., taking or not taking).

FIG. 17 shows a steroid use screen that presents a query similar to FIGS. 11 to 16. However, unlike the queries and the potential answers of FIGS. 11 to 16, the query and the potential answers of FIG. 17 relate to a query relating to a steroid use parameter of the patient. If the patient has not been taking steroids, then a selection of the taking answer would suggest a worsening of the patient's health condition, whether this enquiry is related to a baseline or not.

FIG. 18 shows an inhaler use screen that presents a query similar to FIGS. 11 to 17. However, unlike the queries and the potential answers of FIGS. 11 to 17, the query and the potential answers of FIG. 18 relate to a query relating to an inhaler use parameter of the patient. If the patient's doctor sets a baseline of, for example, 1 to 3 times per day, then anything to the left of that answer would suggest a worsening of the patient's health condition, whether this enquiry is related to a baseline or not.

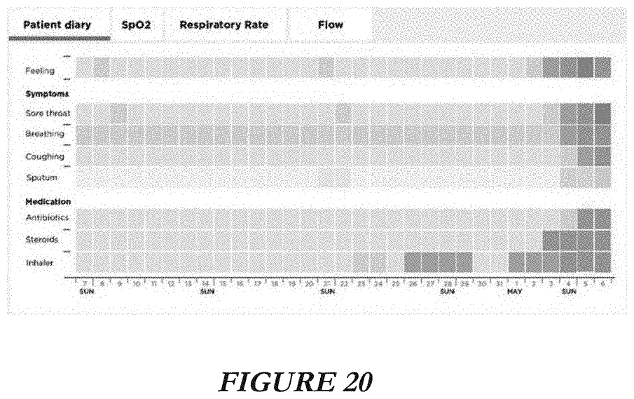

FIG. 21 also shows a number of queries for example related to the patient breathing, patient tiredness, patient throat and muscles, where the patient has a temperature and/or shivers, and patient infection.

FIG. 19 shows a patient and device management platform based (e.g. a server based) dashboard presenting a plot formed based a plurality of answers to a plurality of queries of a personal health enquiry.

FIG. 20 shows the plot of FIG. 19 tracking the answers against time based on a plurality of symptom criteria and a plurality of medication criteria. The patient's doctor (or another health provisional or healthcare provider) may operating a computing device, as explained above, and thereby access the data relating to the patient's answers through an application as explained above.

FIG. 20 may show the response to each query relating to the specific health parameter, or may show the overall condition of a health parameter based on multiple queries. It will be appreciated that where it is described in the specification as relating to showing a response it may also be showing a condition of a health parameter (based on for example a number of responses, and/or one or more patient parameters).

For each of his or her patients (e.g., based on patient or machine identifier), a doctor may operate the computing device and thereby view the server-based dashboard. On this webpage, the doctor may view a chart (Detail A) that tracks the patient's responses to the enquiry.

The information relating to the patient's responses to the enquiry over time, and/or any information on patent parameter over time may be shown as a report (for example a patient diary). The report may comprise one or more chart (as described below), and information on patient parameters and parameters of the apparatus The report may also comprise information on for example any alarms, compliance, usage, or the prescription for the patient.

The report may also indicate the time a prescription has changed so that the health provider can see the effect of this on one or more health parameters.

The report may allow for a health provider to assess whether the prescription is effective. For example clinician may reduce flow rate required if patient is improving. Alternatively, for example the health provider may increase flow or 02 concentration if symptoms are worsening.

In some embodiments, based on the report the clinician may also increase humidity if there is reduced compliance.

The report may be a daily report, i.e. the report is generated daily or the responses to the queries plotted on a daily basis.

The report may show the responses to the enquiry for each day (for example as shown in FIGS. 20 and 21. If the patient answers an enquiry multiple times a day the worst determination for each health parameter, or response may be displayed, or an average of the responses may be taken.

The report can also provide an indication to the clinician if a patient does not complete the enquiry for a particular day or days. The days the enquiry is not completed is presented in the report, and for example as shown in FIG. 21 may comprise a blank cell. The indication an enquiry has not been completed on a particular day can also provide a warning to the clinician that the patient requires assistance or can prompt clinician to investigate. For example if there are several days with no completed enquiry, this can indicate patient may have been admitted to hospital.

The report may group one or more health parameters (for example as shown in FIG. 21).

In some embodiments, one or more notifications may be raised if the enquiry has not been completed for a predetermined number of days in a row, or a predetermined number of days in a time period (for example 4 times in a week)

This chart (FIG. 19) allows the doctor to (among other things): track aspects of the patient's health, consider whether they think the patient's medical state is going to deteriorate to the point of hospitalization, and monitor the nasal high flow device's predictions of whether the patient's medical state is going to deteriorate to the point of hospitalization.

FIG. 21 shows an alternative plot of FIG. 20 tracking the answers against time based on a plurality of symptom criteria and a plurality of medication criteria In FIG. 21 each of the patients responses may be shown with an associated numerical score (as also described below) to indicate the response relative to other responses. For example the more negative responses may be numbered higher relative to more positive responses. The relative numerical score may allow a healthcare professional (for example a doctor) to more easily track aspects of the patient's health, consider whether they think the patient's medical state is going to deteriorate to the point of hospitalization, and monitor the nasal high flow device's predictions of whether the patient's medical state is going to deteriorate to the point of hospitalization

The numerical score may be provided additionally to colour coding (as shown in FIG. 21), or alternatively.

As illustrated in FIGS. 20 and 21 the colour coding may indicate that the associated health parameter is worsening. The colour coding may range from a first colour related to no change or minimal change in the response (for example a grey or blue) to a second colour related to a substantial worsening in the response (a dark red). It will be appreciated that multiple intermediate colours may be provided between the first colour and second colour relating to varying levels of worsening (for example as shown in Figure where there is a blue which indicates minor worsening, and a orange which indicates major worsening).

In some embodiments the colour coding may include some colours which indicate an improvement in the health parameter. For example if the responses indicate a health parameter is improving the colour green may indicate the associated health parameter is improving.

For example for a particular day if the response is worse than the previous day (or an average of the previous number of days) the colour may be a darker red.

The plot for example as shown in FIGS. 19, 20 and 21 may also show one or more patient parameters, for example SpO2 and respiratory rate, as described in more detail above. The one or more patient parameters may be shown against time.

Additionally or alternatively, the plot for example as shown in FIGS. 19, 20 and 21 may also show compliance information indicating the compliance of the patient with therapy (which may for example include usage time).

Compliance information may be determined based on the detection of breathing of the nasal high flow system. Compliance may be determined by detection of breathing of a patient based on a flow signal for example as described in PCT/IB2020/051816. The contents of that specification are incorporated herein in its entirety by way of reference

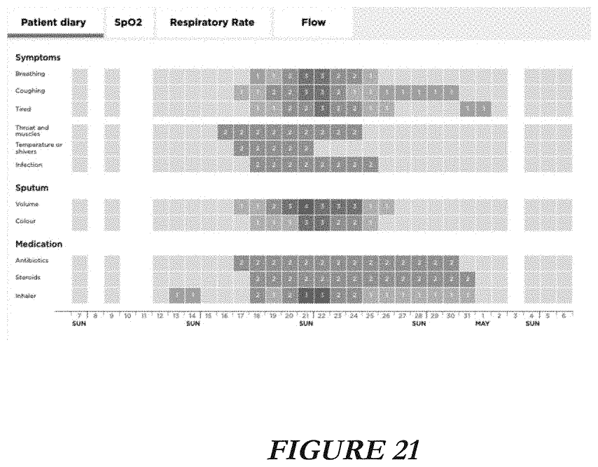

FIG. 22 shows a health provider deterioration overview.

The deterioration overview may display the number of patients with certain health parameters being above a threshold which may be indicative of a patient deteriorating (for example dark green sputum).

The deterioration overview may also display the number of patients who are deteriorating.

The deterioration overview may also display the number of patients and the length of time the patients have been deteriorating.

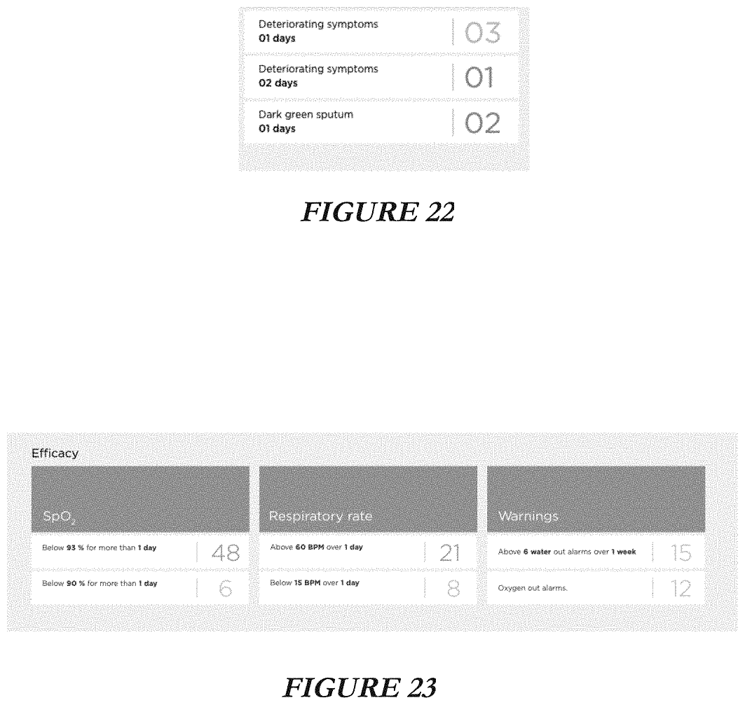

FIG. 23 shows an efficacy overview. The efficacy overview may also display various patient parameters (for example SpO2, respiratory rate) and group patients based on a threshold.

The efficacy overview may also display other warnings from the apparatus (for example water out alarms, or oxygen out alarms).

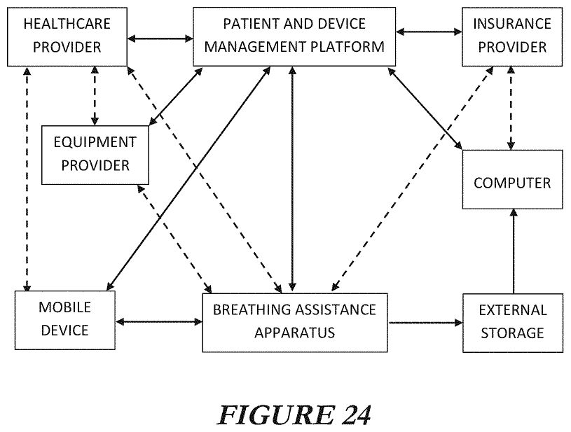

Shown in FIG. 24 is an architecture diagram showing a system for providing breathing assistance or providing respiratory therapy to a patient. Briefly stated, the breathing assistance apparatus produces data in the form of patient answers to the one or more queries of the enquiry. In one embodiment the breathing assistance apparatus may additionally produce data in the form of a dashboard and/or plot as shown in FIGS. 19 and 20 respectively. The data (answers and/or dashboard and/or plot) may then be provided to an external storage device such as a USB, a patient and device management platform, a mobile device (e.g. smartphone, laptop, tablet, wearable), insurance provider or equipment provider. If the data is provided to a USB, then that data may later be downloaded into a computer, that can then feed the data to the patient and device management platform, or insurance provider. In some embodiments the mobile device or patient and device management platform may be able to provide data back to the breathing assistance apparatus (e.g. information to the patient about a change in a physiological condition or a pathology of that patient).

A healthcare provider may receive data about the patient (i.e. their answers to the queries, and/or dashboard and/or plot) via a mobile device (e.g. smartphone, laptop, tablet, wearable), directly from the breathing assistance apparatus, the patient and device management platform or from an equipment provider. The healthcare provider may be able to then feed data (e.g. information to the patient about a change in a physiological condition or a pathology of that patient) back to the breathing assistance apparatus (e.g. via the patient and device management platform) or may actively modify the treatment prescription of the breathing assistance apparatus.

As described above, a user of the breathing assistance apparatus is provided an enquiry, formed of one or more queries, on an integrated touchscreen of a breathing assistance apparatus for providing therapy (“breathing assistance apparatus”). The enquiry is received on one or more touch screens, through which the user also provides their response. The responses are plotted in a graphical format that visually illustrates the health parameters recorded by the patient

As shown in FIG. 7, in some embodiments a baseline 5902 of health status for a patient is first established. For example, this baseline status of the patient's health parameters may be established independently by a health care provider, or established by the patient answering one or more queries of an enquiry from the apparatus. In such an embodiment where a baseline status of a patients heath parameters is established, a healthcare provider (and/or the patient) may be able to determine a change from baseline, whether that be an improvement in one or more health parameters or a worsening in one or more health parameters. In one embodiment the specific baseline for a particular patient may be stored locally on the breathing assistance apparatus or may be accessed from a remote device (e.g. a server).

Each health parameter may comprise an associated baseline.

The baseline for a health parameter may be dynamically adjusted (for example by the apparatus and/or patient and device management system).

The baseline may be updated based on the answers to one or more queries as part of the health enquiry over a period of time.

In some embodiments the baseline for a health parameter may be updated based on a period of consistent answers to a query or enquiry by patient. For example the baseline for a health parameter may be updated if the answers to the query or queries related to the health parameter are all the same or within a predetermined deviation threshold of the baseline.

The baseline may be updated if, the answers to the query or queries related to the health parameter, indicate a different baseline. The baseline may be updated to the different baseline if based on the answers to the query or queries related to the health parameter indicate a different baseline over a predetermined time (for example number of days) or number of responses to the health enquiry.

In some embodiments, the baseline may be iterated towards the different baseline

For example if the patient consistency answers that they have an occasional cough (i.e. FIG. 14) ten times in a row, then the baseline associated with this patient parameter may be updated to be an occasional cough.

The baseline may be updated based on the season. For example if the season is winter the patient's symptoms will be expected to be worse and the worsening of symptoms will be reflected in the answers to the queries (for example they will be negative).

The baseline may be updated to be lower or more negative in colder seasons (for example winter). The baseline may be updated to be higher or more positive in warmer seasons (for example

The baseline may be updated based on weather or weather forecasts.

The baseline may be updated to be higher or more positive if the weather is warmer, or the humidity is higher. The baseline may be updated to be lower or more negative if the weather is colder, or the humidity is lower.

The baseline may be updated based on the location of the apparatus.

The weather, and/or season and/or location of the apparatus may be entered by a patient (or other user when setting up the apparatus), or may be determined by location of sale, or by a location system of the apparatus (for example GPS, GNNS, or cell tower triangulation).

In some embodiments, before the baseline is updated the patient may be prompted as to whether they would like the baseline to be updated.

In some embodiments, before the baseline is updated the healthcare provide (for example a doctor) may be prompted as to whether they would like the baseline to be updated.

The baseline may be updated periodically (for example once every 6 months).

Once the breathing assistance apparatus is started, the enquiry is displayed on the touch screen for the patient to answer 5904. As explained above, the enquiry can comprise one or more queries and can be displayed on one or more screens. The apparatus then receives an answer for each of the queries from the patient 5906.

As shown in FIGS. 19 and 20, the responses may be plotted in a graphical format that visually illustrates the health parameters recorded by the patient. In one embodiment the plotted health parameters allows a health provider to more easily review the health status of the user (patient). Thus the plotted data (i.e. visual presentation of the responses to the enquiry) allows a healthcare provider to track a change in the health parameters and determine a health condition or change in the health condition of the patient.

In some embodiments the determination of an exacerbation or the onset of an exacerbation (as described above) may be provided to the user.

As shown in FIG. 23, a healthcare provider may access the data relating to the patient's answers. This access may be via a direct provision of data to a healthcare provider or via an equipment provider, a patient and device management platform, or server based application. Additionally the access may be via a mobile device such as a phone.

In some embodiments the patient may skip the enquiry if, for example, they are feeling too unwell to complete it. In some embodiments the breathing assistance apparatus may require the patient to provide answers to each of the queries of the enquiry before allowing them to access the therapy control screen, from where they can initiate therapy.

The answers given by the patient to the enquiry may be used by a healthcare provider to determine (e.g., remotely, locally) a change in a health parameter (e.g., worse, same, better) of the patient. For example, the answers given by the patient to the enquiry can be used to ascertain inputs related to specific health parameters or physiological parameters.

The enquiry may provide queries with selectable graphics corresponding to answers to the queries. The patient can touch the selectable graphics to complete the enquiry.

In some embodiments the breathing assistance apparatus sends the patient's answers to the enquiries to a server for plotting the answers.

Alternatively, the breathing assistance apparatus can be configured to plot the patient's answers.

In some embodiments, based on the answer fgiven by a patient to one or more enquiries, the server may alert a computing device (e.g., smartphone, tablet, wearable, workstation) operated by a healthcare provider.

In some embodiments a healthcare provider has access to the patient's answers to the enquiries and is able to use those answers, whether from a single session or multiple sessions of answers to enquires, to do any one or more of the following.
Change the patient's treatment prescription provided by the breathing assistance apparatus.
Customize the enquiries presented to that patient.
Prescribe a pharmacological intervention.

In some embodiments, based on the patient's answers to the enquiries, from a single session or multiple sessions of answers to enquires, the apparatus and/or the patient and device management platform is configured to contact a healthcare provider (for example a doctor) or alert an emergency service (for example an ambulance service).

In some embodiments, based on the determination that the patient is having an onset of an exacerbation the apparatus and/or the patient and device management platform is configured to contact a healthcare provider (for example a doctor) or alert an emergency service (for example an ambulance service).

Preferably the healthcare provider bases their action on the plotted results of the patient's health condition.

In relation to changing a patient's treatment prescription, the healthcare provider may receive the answers to the queries provided by the patient, and upon reviewing those answers remotely change one or more treatment settings (the prescription) of the breathing assistance apparatus. Such as, the flow rate, percentage O2, treatment pressure, and/or duration of treatment.

The healthcare provider may customize the queries that are provided to the patient. For example, the query may be customised to a patient to more effectively provide health care parameters targeted to their health needs, or the queries may be varied to maintain the patient's interest (for example as custom queries as described above).

Based on the answers provided by the patient to the queries, the healthcare provider may prescribe pharmacological interventions such as prescribing antibiotics, steroids and/or inhaler use.

In some embodiments the queries may change as the queries asked may be dependent on the answers given previously (in the same session or in previous sessions).For example, the controller may be configured to selectively present particular queries based on the previous responses of the patient or based on a change in one or more health parameters over time or a change in the health condition of the patient over time.

The healthcare provider may determine if a patient's health condition is going to deteriorate based on the plotted results of the patient's medical condition. Alternatively, the change in a patient's health condition may be presented on the screen of the device or an alarm may be generated by the breathing assistance apparatus to indicate to a user that the patient needs to visit a doctor or hospital.

In one embodiment, making completion of the enquiry mandatory ensures that that patient heath information is gathered. For example, the breathing assistance apparatus may be configured to require patient answers to the enquiry mandatory. Use of the breathing assistance apparatus may be locked (e.g., flow rate or humidity level may be locked until the patient provides their answers to the enquiry). Preventing use of the breathing assistance apparatus will ensure a user of the breathing assistance apparatus completes the enquiry and healthcare providers (e.g., patient's doctor) and/or patient can react in a timely manner.

In some embodiments, the mandatory completion of the enquiry can be relaxed or adjusted. For example, so that the enquiry need only be completed once per predetermined interval (for example, such as once per day). There may be other situations warranting bypassing of enforcing the mandatory completion of the enquiry. For example, if a patient's answer to an enquiry indicates a need to start receiving therapy immediately. Furthermore, the mandatory completion of the enquiry may be adjusted if the answers provided by the user indicate that the user is providing false or inconsistent answers to the enquiries. For example, this may be determined where inconsistent answers are provided to related enquiries. In some embodiments this information is also represented graphically.

The mandatory completion of the enquiry can be enforced by preventing effective operation of the breathing assistance apparatus until responses to a predetermined set of enquiries have been received. Requiring patients to complete the enquiry before every therapy session may increase the likelihood of patients regularly completing the enquiry.

As described above, the enquiry can include a range of queries. The queries can be general queries directed to a patient's physical or mental health. For example, as shown in FIG. 11 is a general query asking “How are you feeling this morning?” along with three options “better”, “usual” and “worse”. The patient is free to select one of these options on the touch screen. This type of query is a subjective qualitative query. That is, different patients may answer this query differently despite feeling the same. When compared to a baseline the answer to this query provides a comparative reference point for the patient's general feeling.

As shown in FIGS. 12 and 13 are further queries asking “Do you have a sore throat?” and “How is your breathing?”, along with five different options for answering being from “extremely” through to “not at all” and “difficult” to “easy” respectively. Again, self reporting degrees of pain and/or discomfort are subjective but provide a useful comparison when compared to a baseline. The scoring system is made easy to use such as in FIGS. 11 and 12 using colourful diagrams, and by representing the score pictorially (in this case with a face showing degrees of happiness or displeasure).

FIG. 14 shows a query asking “Are you still coughing?” with five different options ranging from “all of the time” though to “not at all”. This query is a subjective quantitative query that it reports the frequency of something but is subject to a patient's subjective analysis of their degree of coughing.

FIG. 15 shows a query asking “What colour is your sputum?” and then provides a colour chart of five colours to which a patient can compare their sputum. Use of a colour chart for analyzing sputum is known (see Stockley R A, Bayley D, Hill S L, Hill A T, Crooks S, Campbell E J. Assessment of airway neutrophils by sputum colour: correlation with airways inflammation. Thorax 2001; 56: 366-372). Thus, this type of query provides an objective qualitative assessment of a heath parameter.

FIGS. 16 to 17 show quantitative queries. That is the patient provides objective quantitative answers. The queries include “are you taking antibiotics?”, or “Are you taking steroids?” for which they can select “taking” or “not taking”. Shown in FIG. 17 is the query “Are you using you inhaler?” which provides for four qualitative answers.

The screen 212 may be configured to present a first query to a user, the first query relating to how a user is feeling and presenting a plurality of discrete selectable responses. The screen 212 may then be configured to present a second query to a user, the second query related to, for example, the condition of a user's throat and the screen 212 presents a plurality of selectable indicia, each indicia representing (or corresponding) to a response. The screen 212 may be configured to present a third query related to a breathing or breathing rate of a patient.

In some embodiments the queries are asked consecutively.

Alternatively, in some embodiments the queries that are asked depend on the answers given to previous queries, whether in the same session or previous sessions.

For example, if a query asks about the colour of sputum, and the answer given indicates a mucopurulent sputum, then follow up queries may target the amount, timing and duration of antibiotic use.

Likewise, if an answer to a query indicates that the patients' health parameters are positive, then certain queries may be skipped.

In one embodiment each answer provided by a patient to a query is represented by a numerical score, and the cumulative total is used to initiate an event. The event may be selected from sending of data to a healthcare provider, indicating sent data as urgent or non-urgent, and sending the data to particular devices.

For example, a “positive” answer may be given a score of 1 or 2, with a higher score of 4 or 5 represents a negative answer (positive and negative here being relative to a health condition). Therefore, a series of low scores may not trigger a threshold event such as sending the data to a health care provider. However, if a patient is moderately unwell then a fist threshold may be met which sends the data to a healthcare provider. If the patent is particularly unwell as indicate by a high score, then that data may be sent with an alert, and/or sent to a healthcare provider's mobile device for urgent attention.

The present disclosure enables an engaging, easy-to-use enquiry that is displayed on the touch screen of a breathing assistance apparatus

To enable timely and informed use of the responses to the enquiry, it is helpful for the responses to be processed either locally on the breathing assistance apparatus or remotely, and then made available in a useable format. For example, the enquiry can be presented on the integrated touchscreen upon start up of the breathing assistance apparatus before any other selectable options are presented to the user.

The presented enquires may be intuitive to engage the user and capture the user's attention. Since the patient is already accustomed to using the breathing assistance apparatus for receiving therapy, the patient is more likely to complete the answers to the queries of the enquiry. Further, making the enquiry intuitive and engaging encourages the patient to regularly interact with the enquiry, while ensuring that the enquiry is not overly tedious to complete.

The enquiry being presented on the integrated touchscreen of the apparatus also makes the enquiry easier to access since the user does not need to start or handle a second device (e.g. a phone). This makes the user more likely to complete the enquiry due to easier use (i.e. a single device for therapy and answering queries).

Reference to any prior art in this specification is not, and should not be taken as, an acknowledgement or any form of suggestion that the prior art forms part of the common general knowledge in the field of endeavour in any country in the world.

Where reference is used herein to directional terms such as ‘up’, ‘down’, ‘forward’, ‘rearward’, ‘horizontal’, ‘vertical’ etc, those terms refer to when the apparatus is in a typical in-use position and/or with reference to particular orientations shown in the figures, and are used to show and/or describe relative directions or orientations.

1.-110. (canceled)

111. A breathing assistance apparatus comprising:
a housing;
a source of breathable gas or gases;
a breathing conduit that conveys the breathable gas to a patient via a patient interface;
a touchscreen located on the housing;
a controller located within the housing to control the breathing assistance apparatus operations, including activation and operation of the breathing assistance apparatus,
wherein the controller is configured to:
request the touchscreen to display a plurality of user health queries and a plurality of user input elements via which user inputs are received; and
refrain from activating or prevent activation of a component of the apparatus until a predetermined plurality of the user inputs have been received.

112. The apparatus of claim 111, further comprising a flow generator, a humidifier located within the housing downstream of and in fluid communication with the flow generator and upstream of and in fluid communication with a fluid outlet, the humidifier including a heater plate, and wherein the controller is in electrical communication with, to control the activation and operation of, the flow generator and the heater plate.

113. The apparatus of claim 112, wherein the controller is configured to request the touchscreen to display a page after the controller receives the user inputs, the page configured to present a menu to control or activate the flow generator or the humidifier or to input an operational parameter of the flow generator or the humidifier.

114. The apparatus of claim 111, wherein the controller is configured to request the touchscreen to display the user health queries and the plurality of user input elements such that at least one of the user health queries or at least one of the plurality of user input elements is different between at least two instances of a flow generator being activated over a predetermined time period.

115. The apparatus of claim 111, further comprising a network interface, located in the housing and in electrical communication with the controller, the network interface configured to send the predetermined plurality of the user inputs to an external or remote service or platform.

116. The apparatus of claim 115, wherein the controller is configured to request the network interface to send the predetermined plurality of user inputs to a patient and device management platform one-by-one after each of the user inputs is received via the controller.

117. The apparatus of claim 115, wherein the controller is configured to request the network interface to send the predetermined plurality of user inputs to a patient and device management platform on a group-basis after a group of the user inputs are received via the controller.

118. The apparatus of claim 116, wherein the network interface is configured to receive a message from the patient and device management platform, the message being based on the predetermined plurality of the user inputs sent to the patient and device management platform via the network interface.

119. The apparatus of claim 118, wherein the message includes video content for output via the touchscreen.

120. The apparatus of claim 118, wherein the network interface is a first network interface and the message is a first message, the apparatus further comprising a second network interface located in the housing, the second network interface configured to send a second message to a computing device responsive to the first message, the second message being associated with the predetermined plurality of user inputs sent to the patient and device management platform via the first network interface, the second network interface being local to the computing device, the computing device is other than the patient and device management platform.

121. The apparatus of claim 118, wherein the message is informative of a user health parameter worsening over a predetermined time period as determined based on at least one of the predetermined plurality of the user inputs.

122. The apparatus of claim 121, wherein the message is informative of the user health parameter worsening relative to a baseline.

123. The apparatus of claim 121, wherein the message is informative that two or more health parameters are worsening for at least two days and the patient is therefore deteriorating.

124. The apparatus of claim 115, wherein the controller is configured to allow skipping of the user health queries before activating a flow generator, as the component of the apparatus, such that the user health queries that are skipped form a plurality of data points that are sent to a patient and device management platform via the network interface.

125. The apparatus of claim 111, wherein the user health queries include a first query and a second query, wherein the first query precedes the second query, wherein the user inputs include a first user input and a second user input, wherein the first user input precedes the second user input, wherein the first user input corresponds to the first query, wherein the second user input corresponds to the second query, wherein the second query is content-dependent on the first user input.

126. The apparatus of claim 125, wherein the first user input and the second user input are from a same user session.

127. The apparatus of claim 125, wherein the first user input is from a first user session, wherein the second user input is from a second user session, wherein the first user session precedes the second user session.

128. The apparatus of claim 111, wherein the controller is configured to prevent activation of a flow generator, as the component of the apparatus, until a predetermined plurality of the user inputs have been received.

129. The apparatus of claim 111, wherein at least one of the user health queries relates to a user disease progression or a user health condition.

130. The apparatus of claim 111, wherein at least two of the plurality of user input elements are visually distinct from each other based on one or more of: shape, size, color or shade of color.
